SEQUENCE LISTING (39 sequences)

SEQ ID NO: 1 - protein, 13 aa, Artificial Sequence
  Synthetic Construct
KASGYTFTDYYIN

SEQ ID NO: 2 - protein, 17 aa, Artificial Sequence
  Synthetic Construct
WINPGSGNTKYNEKFKG

SEQ ID NO: 3 - protein, 8 aa, Artificial Sequence
  Synthetic Construct
TREGETVY

SEQ ID NO: 4 - protein, 16 aa, Artificial Sequence
  Synthetic Construct
KSSQSLLYSRGKTYLN

SEQ ID NO: 5 - protein, 8 aa, Artificial Sequence
  Synthetic Construct
YAVSKLDS

SEQ ID NO: 6 - protein, 8 aa, Artificial Sequence
  Synthetic Construct
YDVSKLDS

SEQ ID NO: 7 - protein, 9 aa, Artificial Sequence
  Synthetic Construct
VQGTHYPFT

SEQ ID NO: 8 - protein, 115 aa, Artificial Sequence
  Synthetic Construct
QVQLVQSGAEVKKPGSSVKVSCKASGYTFTDYYINWVRQAPGQGLEWMGWINPGSGNTKYNEKFKGRVTITADESTSTAYMELSSLRSEDTAVYYCTREGETVYWGQGTLVTVSS

SEQ ID NO: 9 - protein, 112 aa, Artificial Sequence
  Synthetic Construct
DVVMTQSPLSLPVTLGQPASISCKSSQSLLYSRGKTYLNWFQQRPGQSPRRLIYDVSKLDSGVPDRFSGSGSGTDFTLKISRVEAEDVGVYYCVQGTHYPFTFGQGTKLEIK

SEQ ID NO: 10 - protein, 112 aa, Artificial Sequence
  Synthetic Construct
DIQMTQSPSTLSASVGDRVTITCKSSQSLLYSRGKTYLNWLQQKPGKAPKLLIYAVSKLDSGVPSRFSGSGSGTEFTLTISSLQPDDFATYYCVQGTHYPFTFGQGTKLEIK

SEQ ID NO: 11 - protein, 444 aa, Artificial Sequence
  Synthetic Construct
QVQLVQSGAEVKKPGSSVKVSCKASGYTFTDYYINWVRQAPGQGLEWMGWINPGSGNTKYNEKFKGRVTITADESTSTAYMELSSLRSEDTAVYYCTREGETVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 219 aa, Artificial Sequence
  Synthetic Construct
DVVMTQSPLSLPVTLGQPASISCKSSQSLLYSRGKTYLNWFQQRPGQSPRRLIYDVSKLDSGVPDRFSGSGSGTDFTLKISRVEAEDVGVYYCVQGTHYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 219 aa, Artificial Sequence
  Synthetic Construct
DIQMTQSPSTLSASVGDRVTITCKSSQSLLYSRGKTYLNWLQQKPGKAPKLLIYAVSKLDSGVPSRFSGSGSGTEFTLTISSLQPDDFATYYCVQGTHYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - DNA, 1332 bp, Artificial Sequence
  Synthetic Construct
caggtgcagctggtgcagtctggggctgaggtgaagaagcctgggtcctcggtgaaggtctcctgcaaggcttctggatacaccttcaccgactattatatcaactgggtgcgacaggcccctggacaagggcttgagtggatgggatggatcaaccctggcagtggtaatacaaagtacaatgagaagttcaagggcagagtcacgattaccgcggacgaatccacgagcacagcctacatggagctgagcagcctgagatctgaggacacggccgtgtattactgtacaagagaaggcgagacggtctactggggccagggaaccctggtcaccgtctcctcagcctccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgccccccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 15 - DNA, 657 bp, Artificial Sequence
  Synthetic Construct
gatgttgtgatgactcagtctccactctccctgcccgtcacccttggacagccggcctccatctcctgcaagtctagtcaaagcctcctgtacagtcgcggaaaaacctacttgaattggtttcagcagaggccaggccaatctccaaggcgcctaatttatgatgtttctaaactggactctggggtcccagacagattcagcggcagtgggtcaggcactgatttcacactgaaaatcagcagggtggaggctgaggatgttggggtttattactgcgtgcaaggtacacactaccctttcacttttggccaagggaccaagctggagatcaaacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 16 - DNA, 657 bp, Artificial Sequence
  Synthetic Construct
gacatccagatgacccagtctccttccaccctgtctgcatctgtaggagacagagtcaccatcacttgcaagtccagtcagagtctcctgtacagtcgcggaaaaacctatttgaactggctccagcagaaaccagggaaagcccctaagctcctgatctatgctgtctccaaactggacagtggggtcccatcaaggttcagcggcagtggatctgggacagaattcactctcaccatcagcagcctgcagcctgatgattttgcaacttattactgcgtgcagggtacacattatcctttcacttttggccaggggaccaagctggagatcaaacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 17 - protein, 16 aa, Artificial Sequence
  Synthetic Construct
KSSQSLLYSRGKTYLN

SEQ ID NO: 18 - protein, 7 aa, Artificial Sequence
  Synthetic Construct
AVSKLDS

SEQ ID NO: 19 - protein, 9 aa, Artificial Sequence
  Synthetic Construct
VQGTHYPFT

SEQ ID NO: 20 - protein, 10 aa, Artificial Sequence
  Synthetic Construct
GYDFTRYYIN

SEQ ID NO: 21 - protein, 10 aa, Artificial Sequence
  Synthetic Construct
GYTFTRYYIN

SEQ ID NO: 22 - protein, 17 aa, Artificial Sequence
  Synthetic Construct
WINPGSGNTKYNEKFKG

SEQ ID NO: 23 - protein, 6 aa, Artificial Sequence
  Synthetic Construct
EGITVY

SEQ ID NO: 24 - protein, 6 aa, Artificial Sequence
  Synthetic Construct
EGTTVY

SEQ ID NO: 25 - protein, 112 aa, Artificial Sequence
  Synthetic Construct
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIK

SEQ ID NO: 26 - protein, 115 aa, Artificial Sequence
  Synthetic Construct
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSS

SEQ ID NO: 27 - protein, 115 aa, Artificial Sequence
  Synthetic Construct
QVQLVQSGAEVKKPGSSVKVSCKASGYTFTRYYINWVRQAPGQGLEWMGWINPGSGNTKYNEKFKGRVTITADESTSTAYMELSSLRSEDTAVYYCAREGTTVYWGQGTTVTVSS

SEQ ID NO: 28 - protein, 219 aa, Artificial Sequence
  Synthetic Construct
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 444 aa, Artificial Sequence
  Synthetic Construct
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 444 aa, Artificial Sequence
  Synthetic Construct
QVQLVQSGAEVKKPGSSVKVSCKASGYTFTRYYINWVRQAPGQGLEWMGWINPGSGNTKYNEKFKGRVTITADESTSTAYMELSSLRSEDTAVYYCAREGTTVYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 40 aa, Artificial Sequence
  Synthetic Construct
  MISC_FEATURE (1)..(1): Xaa at position 1 = pyroglutamic acid
XFRHDSGYEVHHQKLVFFAEDVGSNKGAIIGLMVGGVVIA

SEQ ID NO: 32 - protein, 112 aa, Artificial Sequence; identical to SEQ ID NO: 25
  Synthetic
  MISC_FEATURE (1)..(112): This sequence represents LCVR-B12L/R17L/hE8L

SEQ ID NO: 33 - protein, 219 aa, Artificial Sequence; identical to SEQ ID NO: 28
  Synthetic
  MISC_FEATURE (1)..(219): This sequence represents LC-B12L/R17L

SEQ ID NO: 34 - protein, 115 aa, Artificial Sequence
  Synthetic
  MISC_FEATURE (1)..(115): This sequence represents HCVR-hE8L
QVQLVQSGAEVKKPGSSVKVSCKASGYTFTDYYINWVRQAPGQGLEWMGWINPGSGNTKYNEKFKGRVTITADESTSTAYMELSSLRSEDTAVYYCAREGETVYWGQGTTVTVSS

SEQ ID NO: 35 - protein, 444 aa, Artificial Sequence
  Synthetic
  MISC_FEATURE (1)..(444): This sequence represents HC-hE8L
QVQLVQSGAEVKKPGSSVKVSCKASGYTFTDYYINWVRQAPGQGLEWMGWINPGSGNTKYNEKFKGRVTITADESTSTAYMELSSLRSEDTAVYYCAREGETVYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 10 aa, Artificial Sequence
  Synthetic
  MISC_FEATURE (1)..(10): This sequence represents HCDR1-hE8L
GYTFTDYYIN

SEQ ID NO: 37 - protein, 6 aa, Artificial Sequence
  Synthetic
  MISC_FEATURE (1)..(6): This sequence represents HCDR3-hE8L
EGETVY

SEQ ID NO: 38 - protein, 42 aa, Artificial Sequence [sequence omitted]

SEQ ID NO: 39 - protein, 10 aa, Artificial Sequence
  Synthetic Construct
QTLEFLKIPS

(19) United States
(12) Patent Application Publication
(10) Pub. No.: US 2019/0365774 A1
(43) Pub. Date: Dec. 5, 2019

(54) COMBINATION THERAPY OF BACE-1 INHIBITOR AND ANTI-N3PGLU ABETA ANTIBODY

(71) Applicant: Eli Lilly and Company, Indianapolis, IN (US)

(72) Inventors: Dustin James Mergott, Indianapolis, IN (US); Brian Andrew Willis, Carmel, IN (US)

(21) Appl. No.: 16/321,206

(22) PCT Filed: Aug. 11, 2017
(86) PCT No.: PCT/US2017/046480

§ 371 Date: Jan. 28, 2019

Related U.S. Application Data
(60) Provisional application No. 62/376,422, filed on Aug. 18, 2016.

(51) Int. Cl. A61K 31/546; C07K 16/18

(52) U.S. Cl. CPC A61K 31/546; C07B 2200/13; C07K 2317/565; C07K 16/18

(57) ABSTRACT

The present invention provides a method of treating a cognitive or neurodegenerative disease, comprising administering to a patient in need of such treatment an effective amount of a compound of the formula or a pharmaceutically acceptable salt thereof in combination with an effective amount of an anti-N3pGlu Abeta antibody selected from the group consisting of hE8L, B12L, R17L, Antibody I, and Antibody II.

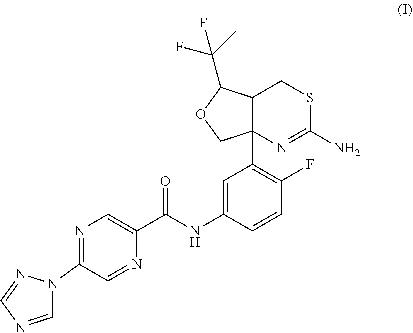 [SMILES: CC(F)(F)C1OCC2(c3cc(NC(=O)c4cnc(-n5cncn5)cn4)ccc3F)N=C(N)SCC12]

The present invention relates to a combination of a BACE inhibitor with an anti-N3pGlu Abeta antibody, and to methods of using the same to treat certain neurological disorders, such as Alzheimer's disease.

The present invention is in the field of treatment of Alzheimer's disease and other diseases and disorders involving amyloid β (Abeta) peptide, a neurotoxic and highly aggregatory peptide segment of the amyloid precursor protein (APP). Alzheimer's disease is a devastating neurodegenerative disorder that affects millions of patients worldwide. In view of the currently approved agents on the market which afford only transient, symptomatic benefits to the patient, there is a significant unmet need in the treatment of Alzheimer's disease.

Alzheimer's disease is characterized by the generation, aggregation, and deposition of Abeta in the brain. Complete or partial inhibition of beta-secretase (beta-site amyloid precursor protein-cleaving enzyme; BACE) has been shown to have a significant effect on plaque-related and plaque-dependent pathologies in mouse models. This suggests that even small reductions in Abeta peptide levels might result in a long-term significant reduction in plaque burden and synaptic deficits, thus providing significant therapeutic benefits, particularly in the treatment of Alzheimer's disease.

Moreover, antibodies that specifically target N3pGlu Abeta have been shown to lower plaque level in vivo (U.S. Pat. No. 8,679,498). N3pGlu Abeta, also referred to as N3pGlu Aβ, N3pE or Abeta_{p3→42}, is a truncated form of the Abeta peptide found only in plaques. Although N3pGlu Abeta peptide is a minor component of the deposited Abeta in the brain, studies have demonstrated that N3pGlu Abeta peptide has aggressive aggregation properties and accumulates early in the deposition cascade.

A combination of a BACE inhibitor with an antibody that binds N3pGlu Abeta peptide is desired to provide treatment for Abeta peptide-mediated disorders, such as Alzheimer's disease, which may be more effective than either drug alone. For example, treatment with such combination may allow for use of lower doses of either or both drugs as compared to each drug used alone, potentially leading to lower side effects while maintaining efficacy. It is believed that targeting the removal of deposited forms of Abeta with an anti-N3pGlu Abeta antibody and a BACE inhibitor will facilitate the phagocytic removal of pre-existing plaque deposits while at the same time reduce or prevent further deposition of Abeta by inhibiting the generation of Abeta.

U.S. Pat. No. 8,278,334 discloses a method of treating a cognitive or neurodegenerative disease comprising administering a substituted cyclic amine BACE-1 inhibitor with an anti-amyloid antibody. WO 2016/043997 discloses a method of treating a disease that is characterized by the formation and deposition of Abeta, comprising a certain BACE inhibitor in combination with an anti-N3pGlu Abeta monoclonal antibody.

Accordingly, the present invention provides a method of treating a cognitive or neurodegenerative disease, comprising administering to a patient in need of such treatment an effective amount of a compound of Formula I:

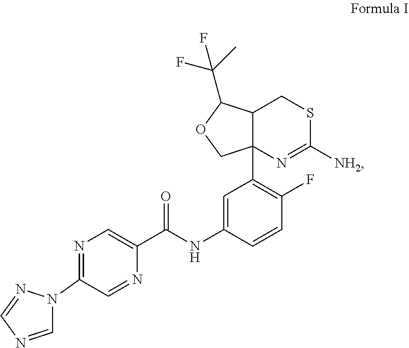 [SMILES: CC(F)(F)C1OCC2(c3cc(NC(=O)c4cnc(-n5cncn5)cn4)ccc3F)N=C(N)SCC12]

or a pharmaceutically acceptable salt thereof, in combination with an effective amount of an anti-N3pGlu Abeta antibody selected from the group consisting of hE8L, B12L, R17L, Antibody I, and Antibody II. The present invention also provides a method of treating a disease that is characterized by the formation and deposition of Abeta, comprising administering to a patient in need of such treatment an effective amount of a compound of Formula I, or a pharmaceutically acceptable salt thereof, in combination with an effective amount of an anti-N3pGlu Abeta antibody selected from the group consisting of hE8L, B12L, R17L, Antibody I, and Antibody II. The present invention further provides a method of treating Alzheimer's disease, comprising administering to a patient in need of such treatment an effective amount of a compound of Formula I, or a pharmaceutically acceptable salt thereof, in combination with an effective amount of an anti-N3pGlu Abeta antibody selected from the group consisting of hE8L, B12L, R17L, Antibody I, and Antibody II. The present invention also provides a method of treating mild Alzheimer's disease, comprising administering to a patient in need of such treatment an effective amount of a compound of Formula I, or a pharmaceutically acceptable salt thereof, in combination with an effective amount of an anti-N3pGlu Abeta antibody selected from the group consisting of hE8L, B12L, R17L, Antibody I, and Antibody II. The present invention further provides a method of treating mild cognitive impairment, comprising administering to a patient in need of such treatment an effective amount of a compound of Formula I, or a pharmaceutically acceptable salt thereof, or a pharmaceutically acceptable salt thereof, in combination with an effective amount of an anti-N3pGlu Abeta antibody selected from the group consisting of hE8L, B12L, R17L, Antibody I, and Antibody II. The present invention further provides a method of treating prodromal Alzheimer's disease, comprising administering to a patient in need of such treatment an effective amount of a compound of Formula I, or a pharmaceutically acceptable salt, in combination with an effective amount of an anti-N3pGlu Abeta antibody selected from the group consisting of hE8L, B12L, R17L, Antibody I, and Antibody II. In addition, the present invention provides a method for the prevention of the progression of mild cognitive impairment to Alzheimer's disease, comprising administering to a patient in need of such treatment an effective amount of a compound of Formula I, or a pharmaceutically acceptable salt thereof, in combination with an effective amount of an anti-N3pGlu Abeta antibody selected from the group consisting of hE8L, B12L, R17L, Antibody I, and Antibody II. The present invention further provides a method of treating cerebral amyloid angiopathy (CAA), comprising administering to a patient in need of such treatment an effective amount of a compound of Formula I, or a pharmaceutically acceptable salt thereof, in combination with an effective amount of an anti-N3pGlu Abeta antibody selected from the group consisting of hE8L, B12L, R17L, Antibody I, and Antibody II.

The present invention further provides a method of treating Alzheimer's disease in a patient, comprising administering to a patient in need of such treatment an effective amount of a compound of the Formula I, or a pharmaceutically acceptable salt thereof, in combination with an effective amount of an anti-N3pGlu Abeta antibody wherein the anti-N3pGlu Abeta antibody comprises a light chain variable region (LCVR) and a heavy chain variable region (HCVR), wherein said LCVR comprises LCDR1, LCDR2 and LCDR3 and HCVR comprises HCDR1, HCDR2 and HCDR3 which are selected from the group consisting of:
a) LCDR1 is SEQ ID. NO: 17, LCDR2 is SEQ ID. NO: 18, LCDR3 is SEQ ID. NO: 19, HCDR1 is SEQ ID. NO: 20, HCDR2 is SEQ ID: NO: 22, and HCDR3 is SEQ ID. NO: 23; and
b) LCDR1 is SEQ ID. NO: 17, LCDR2 is SEQ ID. NO: 18, LCDR3 is SEQ ID. NO: 19, HCDR1 is SEQ ID. NO: 21, HCDR2 is SEQ ID. NO: 22, and HCDR3 is SEQ ID. NO: 24;
c) LCDR1 is SEQ ID. NO: 17, LCDR2 is SEQ ID. NO: 18, LCDR3 is SEQ ID. NO: 19, HCDR1 is SEQ ID. NO: 36, HCDR2 is SEQ ID. NO: 22, and HCDR3 is SEQ ID. NO: 37;
d) LCDR1 is SEQ ID. NO: 4, LCDR2 is SEQ ID. NO: 6, LCDR3 is SEQ ID. NO: 7, HCDR1 is SEQ ID. NO: 1, HCDR2 is SEQ ID. NO: 2, and HCDR3 is SEQ ID. NO: 3;
e) LCDR1 is SEQ ID. NO: 4, LCDR2 is SEQ ID. NO: 5, LCDR3 is SEQ ID. NO: 7, HCDR1 is SEQ ID. NO: 1, HCDR2 is SEQ ID. NO: 2, and HCDR3 is SEQ ID. NO: 3.

Furthermore, the present invention provides a compound of Formula I, or a pharmaceutically acceptable salt thereof, for use in simultaneous, separate, or sequential combination with an anti-N3pGlu Abeta antibody selected from the group consisting of hE8L, B12L, R17L, Antibody I, and Antibody II in the treatment of Alzheimer's disease. In addition, the present invention provides a compound of Formula I, or a pharmaceutically acceptable salt thereof, for use in simultaneous, separate, or sequential combination with an anti-N3pGlu Abeta antibody selected from the group consisting of hE8L, B12L, R17L, Antibody I, and Antibody II in the treatment of mild Alzheimer's disease. Further, the present invention provides a compound of Formula I, or a pharmaceutically acceptable salt thereof, for use in simultaneous, separate, or sequential combination with an anti-N3pGlu Abeta antibody selected from the group consisting of hE8L, B12L, R17L, Antibody I, and Antibody II in the treatment of prodromal Alzheimer's disease. The present invention provides a compound of Formula I, or a pharmaceutically acceptable salt thereof, for use in simultaneous, separate, or sequential combination with an anti-N3pGlu Abeta antibody selected from the group consisting of hE8L, B12L, R17L, Antibody I, and Antibody II in preventing the progression of mild cognitive impairment to Alzheimer's disease.

The present invention provides a compound of the Formula I, or a pharmaceutically acceptable salt thereof, for use in simultaneous, separate, or sequential combination with an anti-N3pGlu Abeta wherein the anti-N3pGlu Abeta antibody comprises a light chain variable region (LCVR) and a heavy chain variable region (HCVR), wherein said LCVR comprises LCDR1, LCDR2 and LCDR3 and HCVR comprises HCDR1, HCDR2 and HCDR3 which are selected from the group consisting of:
a) LCDR1 is SEQ ID. NO: 17, LCDR2 is SEQ ID. NO: 18, LCDR3 is SEQ ID. NO: 19, HCDR1 is SEQ ID. NO: 20, HCDR2 is SEQ ID: NO: 22, and HCDR3 is SEQ ID. NO: 23; and
b) LCDR1 is SEQ ID. NO: 17, LCDR2 is SEQ ID. NO: 18, LCDR3 is SEQ ID. NO: 19, HCDR1 is SEQ ID. NO: 21, HCDR2 is SEQ ID. NO: 22, and HCDR3 is SEQ ID. NO: 24;
c) LCDR1 is SEQ ID. NO: 17, LCDR2 is SEQ ID. NO: 18, LCDR3 is SEQ ID. NO: 19, HCDR1 is SEQ ID. NO: 36, HCDR2 is SEQ ID. NO: 22, and HCDR3 is SEQ ID. NO: 37;
d) LCDR1 is SEQ ID. NO: 4, LCDR2 is SEQ ID. NO: 6, LCDR3 is SEQ ID. NO: 7, HCDR1 is SEQ ID. NO: 1, HCDR2 is SEQ ID. NO: 2, and HCDR3 is SEQ ID. NO: 3;
e) LCDR1 is SEQ ID. NO: 4, LCDR2 is SEQ ID. NO: 5, LCDR3 is SEQ ID. NO: 7, HCDR1 is SEQ ID. NO: 1, HCDR2 is SEQ ID. NO: 2, and HCDR3 is SEQ ID. NO: 3, in the treatment of Alzheimer's disease.

The invention further provides a pharmaceutical composition comprising a compound of Formula I, or a pharmaceutically acceptable salt thereof, with one or more pharmaceutically acceptable carriers, diluents, or excipients, in combination with a pharmaceutical composition of an anti-N3pGlu Abeta antibody selected from the group consisting of hE8L, B12L, R17L, Antibody I, and Antibody II, with one or more pharmaceutically acceptable carriers, diluents, or excipients.

The invention also provides a pharmaceutical composition, comprising a compound of the Formula I, or a pharmaceutically acceptable salt thereof, with one or more pharmaceutically acceptable carriers, diluents, or excipients, in combination with a pharmaceutical composition of an anti-N3pGlu Abeta antibody wherein the anti-N3pGlu Abeta antibody comprises a light chain variable region (LCVR) and a heavy chain variable region (HCVR), wherein said LCVR comprises LCDR1, LCDR2 and LCDR3 and HCVR comprises HCDR1, HCDR2 and HCDR3 which are selected from the group consisting of:
a) LCDR1 is SEQ ID. NO: 17, LCDR2 is SEQ ID. NO: 18, LCDR3 is SEQ ID. NO: 19, HCDR1 is SEQ ID. NO: 20, HCDR2 is SEQ ID: NO: 22, and HCDR3 is SEQ ID. NO: 23; and
b) LCDR1 is SEQ ID. NO: 17, LCDR2 is SEQ ID. NO: 18, LCDR3 is SEQ ID. NO: 19, HCDR1 is SEQ ID. NO: 21, HCDR2 is SEQ ID. NO: 22, and HCDR3 is SEQ ID. NO: 24;
c) LCDR1 is SEQ ID. NO: 17, LCDR2 is SEQ ID. NO: 18, LCDR3 is SEQ ID. NO: 19, HCDR1 is SEQ ID. NO: 36, HCDR2 is SEQ ID. NO: 22, and HCDR3 is SEQ ID. NO: 37;
d) LCDR1 is SEQ ID. NO: 4, LCDR2 is SEQ ID. NO: 6, LCDR3 is SEQ ID. NO: 7, HCDR1 is SEQ ID. NO: 1, HCDR2 is SEQ ID. NO: 2, and HCDR3 is SEQ ID. NO: 3;
e) LCDR1 is SEQ ID. NO: 4, LCDR2 is SEQ ID. NO: 5, LCDR3 is SEQ ID. NO: 7, HCDR1 is SEQ ID. NO: 1, HCDR2 is SEQ ID. NO: 2, and HCDR3 is SEQ ID. NO: 3, with one or more pharmaceutically acceptable carriers, diluents, or excipients.

In addition, the invention provides a kit, comprising a compound of Formula I, or a pharmaceutically acceptable salt thereof, and an anti-N3pGlu Abeta antibody selected from the group consisting of hE8L, B12L, R17L, Antibody I, and Antibody II. The invention further provides a kit, comprising a pharmaceutical composition, comprising a compound of Formula I, or a pharmaceutically acceptable salt thereof, with one or more pharmaceutically acceptable carriers, diluents, or excipients, and a pharmaceutical composition, comprising an anti-N3pGlu Abeta antibody selected from the group consisting of hE8L, B12L, R17L, Antibody I, and Antibody II, with one or more pharmaceutically acceptable carriers, diluents, or excipients. As used herein, a “kit” includes separate containers of each component, wherein one component is a compound of Formula I, or a pharmaceutically acceptable salt thereof, and another component is an anti-N3pGlu Abeta antibody selected from the group consisting of hE8L, B12L, R17L, Antibody I, and Antibody II, in a single package. A “kit” may also include separate containers of each component, wherein one component is a compound of Formula I, or a pharmaceutically acceptable salt thereof, and another component is an anti-N3pGlu Abeta antibody selected from the group consisting of hE8L, B12L, R17L, Antibody I, and Antibody II, in separate packages with instructions to administer each component as a combination.

The invention further provides the use of a compound of Formula I, or a pharmaceutically acceptable salt thereof, for the manufacture of a medicament for the treatment of Alzheimer's disease, mild Alzheimer's disease, prodromal Alzheimer's disease or for the prevention of the progression of mild cognitive impairment to Alzheimer's disease wherein the medicament is to be administered simultaneously, separately or sequentially with an anti-N3pGlu Abeta antibody selected from the group consisting of hE8L, B12L, R17L, Antibody I, and Antibody II.

The following compounds are preferred in the combination:

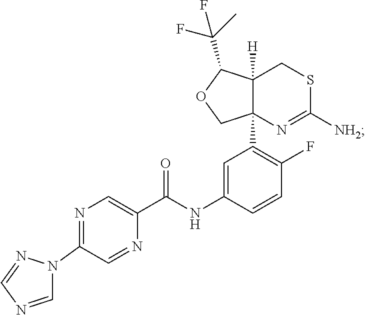 [SMILES: CC(F)(F)[C@H]1OC[C@]2(c3cc(NC(=O)c4cnc(-n5cncn5)cn4)ccc3F)N=C(N)SC[C@H]12]

N-[3-[(4aS,5S,7aS)-2-amino-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide, and the pharmaceutically acceptable salts thereof; and

N-[3-[(4aS,5S,7aS)-2-amino-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide hydrate.

In addition, N-[3-[(4aS,5S,7aS)-2-amino-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide is particularly preferred.

Furthermore, N-[3-[(4aS,5S,7aS)-2-amino-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide malonate; and

N-[3-[(4aS,5S,7aS)-2-amino-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide 4-methylbenzenesulfonate are especially preferred.

The preferred antibodies are hE8L and B12L, R17L, Antibody I, and Antibody II, with hE8L and B12L being especially preferred, and hE8L being most preferred.

The anti-N3pGlu Abeta antibody comprises a light chain variable region (LCVR) and a heavy chain variable region (HCVR), wherein said LCVR comprises LCDR1, LCDR2 and LCDR3 and HCVR comprises HCDR1, HCDR2 and HCDR3 which are selected from the group consisting of:
a) LCDR1 is SEQ ID. NO: 17, LCDR2 is SEQ ID. NO: 18, LCDR3 is SEQ ID. NO: 19, HCDR1 is SEQ ID. NO: 20, HCDR2 is SEQ ID: NO: 22, and HCDR3 is SEQ ID. NO: 23; and
b) LCDR1 is SEQ ID. NO: 17, LCDR2 is SEQ ID. NO: 18, LCDR3 is SEQ ID. NO: 19, HCDR1 is SEQ ID. NO: 21, HCDR2 is SEQ ID. NO: 22, and HCDR3 is SEQ ID. NO: 24;
c) LCDR1 is SEQ ID. NO: 17, LCDR2 is SEQ ID. NO: 18, LCDR3 is SEQ ID. NO: 19, HCDR1 is SEQ ID. NO: 36, HCDR2 is SEQ ID. NO: 22, and HCDR3 is SEQ ID. NO: 37;
d) LCDR1 is SEQ ID. NO: 4, LCDR2 is SEQ ID. NO: 6, LCDR3 is SEQ ID. NO: 7, HCDR1 is SEQ ID. NO: 1, HCDR2 is SEQ ID. NO: 2, and HCDR3 is SEQ ID. NO: 3;
e) LCDR1 is SEQ ID. NO: 4, LCDR2 is SEQ ID. NO: 5, LCDR3 is SEQ ID. NO: 7, HCDR1 is SEQ ID. NO: 1, HCDR2 is SEQ ID. NO: 2, and HCDR3 is SEQ ID. NO: 3.

In other embodiments, the anti-N3pGlu Abeta antibody comprises a light chain variable region (LCVR) and a heavy chain variable region (HCVR), wherein said LCVR and HCVR are selected from the group consisting of:
a) LCVR of SEQ ID NO: 25 and HCVR of SEQ ID NO: 26;
b) LCVR of SEQ ID NO: 25 and HCVR of SEQ ID NO: 27;
c) LCVR of SEQ ID NO: 32 and HCVR of SEQ ID NO: 34;
d) LCVR of SEQ ID NO: 9 and HCVR of SEQ ID NO: 8; and
e) LCVR of SEQ ID NO: 10 and HCVR of SEQ ID NO: 8.

In further embodiments, the anti-N3pGlu Abeta antibody comprises a light chain (LC) and a heavy chain (HC), wherein said LC and HC are selected from the group consisting of:
a) LC of SEQ ID NO: 28 and HC of SEQ ID NO: 29;
b) LC of SEQ ID NO: 28 and HC of SEQ ID NO: 30;
c) LC of SEQ ID NO: 33 and HC of SEQ ID NO: 35;
d) LC of SEQ ID NO: 12 and HC of SEQ ID NO: 11; and
e) LC of SEQ ID NO: 13 and HC of SEQ ID NO: 11.

In further embodiments, the anti-N3pGlu Abeta antibody comprises two light chains (LC) and two heavy chains (HC), wherein each LC and each HC are selected from the group consisting of
a) LC of SEQ ID NO: 28 and HC of SEQ ID NO: 29;
b) LC of SEQ ID NO: 28 and HC of SEQ ID NO: 30;
c) LC of SEQ ID NO: 33 and HC of SEQ ID NO: 35;
d) LC of SEQ ID NO: 12 and HC of SEQ ID NO: 11; and
e) LC of SEQ ID NO: 13 and HC of SEQ ID NO: 11.

In some embodiments, the anti-N3pGlu Abeta antibody comprises hE8L which has a light chain (LC) and a heavy chain (HC) of SEQ ID NOs: 33 and 35 respectively. hE8L further has a light chain variable region (LCVR) and a heavy chain variable region (HCVR) of in SEQ ID NOs: 32 and 34 respectively. The HCVR of hE8L further comprises HCDR1 of SEQ ID NO: 36, HCDR2 of SEQ ID NO: 22 and HCDR3 of SEQ ID NO: 37. The LCVR of hE8L further comprises LCDR1 of SEQ ID NO. 17, LCDR2 of SEQ ID NO. 18 and LCDR3 of SEQ ID NO: 19 respectively.

In some embodiments, the anti-N3pGlu Abeta antibody comprises B12L, which has a light chain (LC) and a heavy chain (HC) of SEQ ID NOs: 28 and 29 respectively. B12L further has a light chain variable region (LCVR) and a heavy chain variable region (HCVR) of SEQ ID NOs: 25 and 26 respectively. The HCVR of B12L further comprises HCDR1 of SEQ ID NO: 20, HCDR2 of SEQ ID NO: 22 and HCDR3 of SEQ ID NO: 23. The LCVR of B12L further comprises LCDR1 of SEQ ID NO. 17, LCDR2 of SEQ ID NO: 18 and LCDR3 of SEQ ID NO: 19 respectively.

In some embodiments, the anti-N3pGlu Abeta antibody comprises R17L which has a light chain (LC) and a heavy chain (HC) of SEQ ID NOs: 28 and 30 respectively. R17L further has a light chain variable region (LCVR) and a heavy chain variable region (HCVR) of SEQ ID NOs: 25 and 27 respectively. The HCVR of R17L further comprises HCDR1 of SEQ ID NO: 21, HCDR2 of SEQ ID NO: 22 and HCDR3 of SEQ ID NO: 24. The LCVR of R17L further comprises LCDR1 of SEQ ID NO. 17, LCDR2 of SEQ ID NO: 18 and LCDR3 of SEQ ID NO: 19 respectively.

In some embodiments, the anti-N3pGlu Abeta antibody comprises Antibody I, which has a light chain (LC) and a heavy chain (HC) of SEQ ID NOs: 12 and 11 respectively. Antibody I further has a light chain variable region (LCVR) and a heavy chain variable region (HCVR) of SEQ ID NOs: 9 and 8 respectively. The HCVR of Antibody I further comprises HCDR1 of SEQ ID NO: 1, HCDR2 of SEQ ID NO: 2, and HCDR3 of SEQ ID NO: 3. The LCVR of Antibody I further comprises LCDR1 of SEQ ID NO: 4, LCDR2 of SEQ ID NO: 6 and LCDR3 of SEQ ID NO: 7 respectively.

In some embodiments, the anti-N3pGlu Abeta antibody comprises Antibody II, which has a light chain (LC) and a heavy chain (HC) of SEQ ID NOs: 13 and 11 respectively. Antibody II further has a light chain variable region (LCVR) and a heavy chain variable region (HCVR) of SEQ ID NOs: 10 and 8 respectively. The HCVR of Antibody II further comprises HCDR1 of SEQ ID NO: 1, HCDR2 of SEQ ID NO: 2, and HCDR3 of SEQ ID NO: 3. The LCVR of Antibody II further comprises LCDR1 of SEQ ID NO: 4, LCDR2 of SEQ ID. NO. 5, and LCDR3 of SEQ ID NO: 7 respectively.

One of ordinary skill in the art will further appreciate and recognize that “anti-N3pGlu Abeta antibody” and the specific antibodies, “hE8L”, “B12L”, and “R17L” are identified and disclosed along with methods for making and using said antibody by one of ordinary skill in the art in U.S. Pat. No. 8,679,498 B2, entitled “Anti-N3pGlu Amyloid Beta Peptide Antibodies and Uses Thereof”, issued Mar. 25, 2014 (U.S. Ser. No. 13/810,895). See for example Table 1 of U.S. Pat. No. 8,679,498 B2. The antibodies, hE8L, B12L, and R17L may be used as the anti-N3pGlu Abeta antibody of the present invention. In other embodiments, the anti-N3pGlu Abeta antibody may comprise the antibody “Antibody I” described herein. In further embodiments, the anti-N3pGlu Abeta antibody may comprise “Antibody II” described herein.

In addition, amino acid sequences for certain antibodies used in the present invention are provided below in Table A:

TABLE A
Antibody SEQ ID NOs

| Antibody | Light | | | |
|---|---|---|---|---|
| Chain | Chain | Heavy | LCVR | HCVR |
| B12L | 28 | 29 | 25 | 26 |
| R17L | 28 | 30 | 25 | 27 |
| hE8L | 33 | 35 | 32 | 34 |
| Antibody I | 12 | 11 | 9 | 8 |
| Antibody II | 13 | 11 | 10 | 8 |

With respect to “hE8L”, “B12L”, “R17L”, “Antibody I”, and “Antibody II”, additional amino acid sequences for such antibodies are provided in Table B:

TABLE B
Additional SEQ ID NOs For “hE8L”, “B12L”, “R17L”,
“Antibody I”, and “Antibody II”
Antibody SEQ ID NOs

| Antibody | LCDR1 | LCDR2 | LCDR3 |
|---|---|---|---|
| B12L | 17 | 18 | 19 |
| R17L | 17 | 18 | 19 |
| hE8L | 17 | 18 | 19 |
| Antibody I | 4 | 6 | 7 |
| Antibody II | 4 | 5 | 7 |

Antibody SEQ ID NOs

| Antibody | HCDR1 | HCDR2 | HCDR3 |
|---|---|---|---|
| B12L | 20 | 22 | 23 |
| R17L | 21 | 22 | 24 |
| hE8L | 36 | 22 | 37 |
| Antibody I | 1 | 2 | 3 |
| Antibody II | 1 | 2 | 3 |

The antibodies of the present invention bind to N3pGlu Aβ. The sequence of N3pGlu Aβ is the amino acid sequence of SEQ ID NO: 31. The sequence of Aβ is SEQ ID NO: 38.

As used herein, an “antibody” is an immunoglobulin molecule comprising two Heavy Chain (HC) and two Light Chain (LC) interconnected by disulfide bonds. The amino terminal portion of each LC and HC includes a variable region responsible for antigen recognition via the complementarity determining regions (CDRs) contained therein. The CDRs are interspersed with regions that are more conserved, termed framework regions. Assignment of amino acids to CDR domains within the LCVR and HCVR regions of the antibodies of the present invention is based on the well-known Kabat numbering convention such as the following: Kabat, et al., Ann. NY Acad. Sci. 190:382-93 (1971); Kabat et al., Sequences of Proteins of Immunological Interest, Fifth Edition, U.S. Department of Health and Human Services, NIH Publication No. 91-3242 (1991)), and North numbering convention (North et al., A New Clustering of Antibody CDR Loop Conformations, Journal of Molecular Biology, 406:228-256 (2011)).

As used herein, the term “isolated” refers to a protein, peptide or nucleic acid that is not found in nature and is free or substantially free from other macromolecular species found in a cellular environment. “Substantially free”, as used herein, means the protein, peptide or nucleic acid of interest comprises more than 80% (on a molar basis) of the macromolecular species present, preferably more than 90% and more preferably more than 95%.

Following expression and secretion of the antibody, the medium is clarified to remove cells and the clarified media is purified using any of many commonly-used techniques. The purified antibody may be formulated into pharmaceutical compositions according to well-known methods for formulating proteins and antibodies for parenteral administration, particularly for subcutaneous, intrathecal, or intravenous administration. The antibody may be lyophilized, together with appropriate pharmaceutically-acceptable excipients, and then later reconstituted with a water-based diluent prior to use. In either case, the stored form and the injected form of the pharmaceutical compositions of the antibody will contain a pharmaceutically-acceptable excipient or excipients, which are ingredients other than the antibody. Whether an ingredient is pharmaceutically-acceptable depends on its effect on the safety and effectiveness or on the safety, purity, and potency of the pharmaceutical composition. If an ingredient is judged to have a sufficiently unfavorable effect on safety or effectiveness (or on safety, purity, or potency) to warrant it not being used in a composition for administration to humans, then it is not pharmaceutically-acceptable to be used in a pharmaceutical composition of the antibody.

The term “disease characterized by deposition of Aβ,” is a disease that is pathologically characterized by Aβ deposits in the brain or in brain vasculature. This includes diseases such as Alzheimer's disease, Down's syndrome, and cerebral amyloid angiopathy. A clinical diagnosis, staging or progression of Alzheimer's disease can be readily determined by the attending diagnostician or health care professional, as one skilled in the art, by using known techniques and by observing results. This generally includes some form of brain plaque imagining, mental or cognitive assessment (e.g. Clinical Dementia Rating-summary of boxes (CDR-SB), Mini-Mental State Exam 25 (MMSE) or Alzheimer's Disease Assessment Scale-Cognitive (ADAS-Cog)) or functional assessment (e.g. Alzheimer's Disease Cooperative Study-Activities of Daily Living (ADCS-ADL). “Clinical Alzheimer's disease” as used herein is a diagnosed stage of Alzheimer's disease. It includes conditions diagnosed as prodromal Alzheimer's disease, mild Alzheimer's disease, moderate Alzheimer's disease and severe Alzheimer's disease.

The term “pre-clinical Alzheimer's disease” is a stage that precedes clinical Alzheimer's disease, where measurable changes in biomarkers (such as CSP Aβ42 levels or deposited brain plaque by amyloid PET) indicate the earliest signs of a patient with Alzheimer's pathology, progressing to clinical Alzheimer's disease. This is usually before symptoms such as memory loss and confusion are noticeable.

As used herein, the terms “treating”, “to treat”, or “treatment”, includes restraining, slowing, stopping, reducing, or reversing the progression or severity of an existing symptom, disorder, condition, or disease.

As used herein, the term “patient” refers to a human.

The term “inhibition of production of Abeta peptide” is taken to mean decreasing of in vivo levels of Abeta peptide in a patient.

As used herein, the term “effective amount” refers to the amount or dose of compound of Formula I, or a pharmaceutically acceptable salt thereof, and to the amount or dose of an anti-N3pGlu Abeta antibody selected from the group consisting of hE8L, B12L, R17L, Antibody I, and Antibody II, which upon single or multiple dose administration to the patient, provides the desired effect in the patient under diagnosis or treatment. It is understood that the combination therapy of the present invention is carried out by administering a compound of Formula I, or a pharmaceutically acceptable salt thereof, together with the anti-N3pGlu Abeta antibody selected from the group consisting of hE8L, B12L, R17L, Antibody I, and Antibody II, in any manner which provides effective levels of the compound of Formula I, and the anti-N3pGlu Abeta antibody selected from the group consisting of hE8L, B12L, R17L, Antibody I, and Antibody II, in the body.

An effective amount can be readily determined by the attending diagnostician, as one skilled in the art, by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

The compound of Formula I, or a pharmaceutically acceptable salt thereof, is generally effective over a wide dosage range in the combination of the present invention. For example, dosages per day of the compound of Formula I normally fall within the range of about 0.1 mg/day to about 500 mg/day, preferably about 0.1 mg/day to about 200 mg/day, and most preferably about 0.1 mg/day to about 100 mg/day. In some embodiments, the dose of the compound of Formula I is about 0.1 mg/day to about 25 mg/day. In addition, the anti-N3pGlu Abeta antibody selected from the group consisting of hE8L, B12L, R17L, Antibody I, and Antibody II is generally effective over a wide dosage range in the combination of the present invention. In some instances dosage levels below the lower limit of the aforesaid ranges may be more than adequate, while in other cases still larger doses may be employed with acceptable adverse events and therefore the above dosage range is not intended to limit the scope of the invention in any way.

The BACE inhibitors and the antibodies of the present invention are preferably formulated as pharmaceutical compositions administered by any route which makes the compound bioavailable. The route of administration may be varied in any way, limited by the physical properties of the drugs and the convenience of the patient and the caregiver. Preferably, anti-N3pGlu Abeta antibody compositions are for parenteral administration, such as intravenous or subcutaneous administration. In addition, the BACE inhibitor compound of Formula I, or a pharmaceutically acceptable salt thereof, is for oral or parenteral administration, including intravenous or subcutaneous administration. Such pharmaceutical compositions and processes for preparing same are well known in the art. (See, e.g., Remington: The Science and Practice of Pharmacy, L. V. Allen, Editor, 22^{nd} Edition, Pharmaceutical Press, 2012).

As used herein, the phrase “in combination with” refers to the administration of the BACE inhibitor, such as the compound of Formula I:

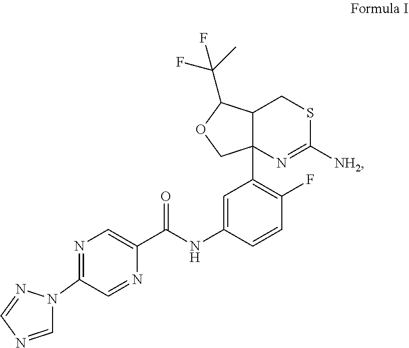 [SMILES: CC(F)(F)C1OCC2(c3cc(NC(=O)c4cnc(-n5cncn5)cn4)ccc3F)N=C(N)SCC12]

or a pharmaceutically acceptable salt thereof, with an anti-N3pGlu Abeta antibody selected from the group consisting of hE8L, B12L, R17L, Antibody I, and Antibody II, simultaneously, or sequentially in any order, or any combination thereof. The two molecules may be administered either as part of the same pharmaceutical composition or in separate pharmaceutical compositions. The compound of Formula I, or a pharmaceutically acceptable salt thereof, can be administered prior to, at the same time as, or subsequent to administration of the anti-N3pGlu Abeta antibody, or in some combination thereof. Where the anti-N3pGlu Abeta antibody is administered at repeated intervals (e.g. during a standard course of treatment), the BACE inhibitor can be administered prior to, at the same time as, or subsequent to, each administration of the anti-N3pGlu Abeta antibody, or some combination thereof, or at different intervals in relation to therapy with the anti-N3pGlu Abeta antibody, or in a single or series of dose(s) prior to, at any time during, or subsequent to the course of treatment with the anti-N3pGlu Abeta antibody.

The compounds of the present invention may be prepared by a variety of procedures known in the art, some of which are illustrated in the Preparations and Examples below. The specific synthetic steps for each of the routes described may be combined in different ways, or in conjunction with steps from different procedures, to prepare compounds of Formula I, or salts thereof. The products of each step can be recovered by conventional methods well known in the art, including extraction, evaporation, precipitation, chromatography, filtration, trituration, and crystallization. In addition, all substituents unless otherwise indicated, are as previously defined. The reagents and starting materials are readily available to one of ordinary skill in the art.

It is understood by one of ordinary skill in the art that the terms “tosylate”, “toluenesulfonic acid”, “p-toluenesulfonic acid”, and “4-methylbenzene sulfonic acid” refer to the compound of the following structure:

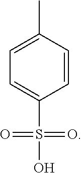

As used herein, “BSA” refers to Bovine Serum Albumin; “EDTA” refers to ethylenediaminetetraacetic acid; “ee” refers to enantiomeric excess; “Ex” refers to example; “F12” refers to Ham's F12 medium; “hr refers to hour or hours; “HRP” refers to Horseradish Peroxidase; “IC₅₀” refers to the concentration of an agent that produces 50% of the maximal inhibitory response possible for that agent; “min” refers to minute or minutes; “PBS” refers to Phosphate Buffered Saline; “PDAPP” refers to platelet derived amyloid precursor protein; “Prep” refers to preparation; “psi” refers to pounds per square inch; “Rₜ” refers to retention time; “SCX” refers to strong cation exchange chromatography; “THF” refers to tetrahydrofuran and “TMB” refers to 3,3′,5,5′-teramethylbenzidine.

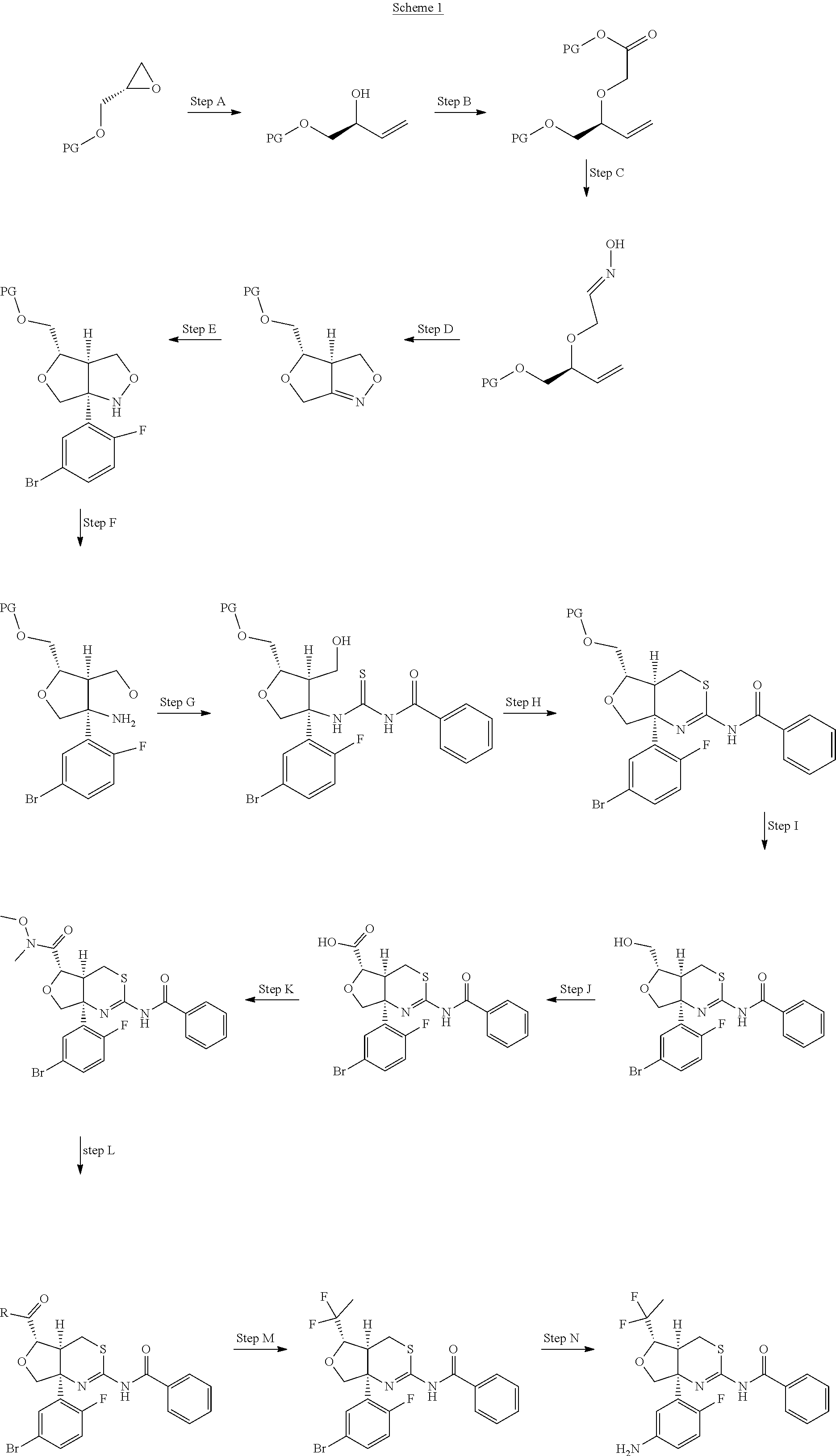

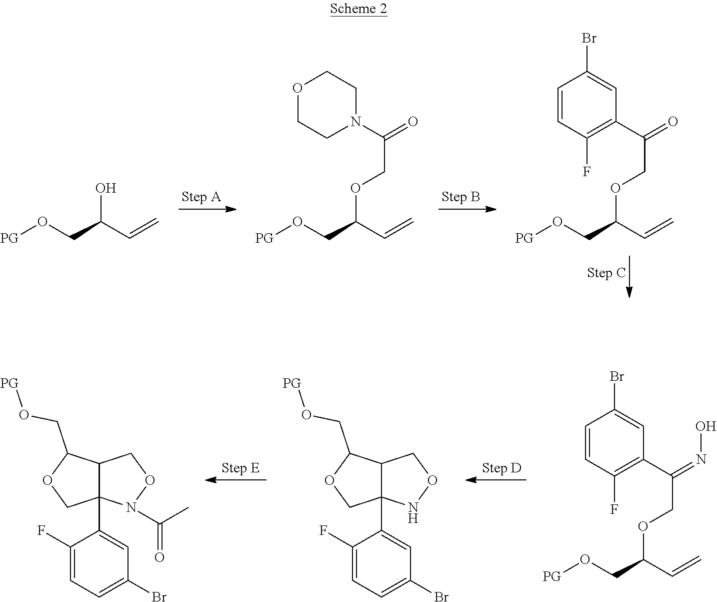

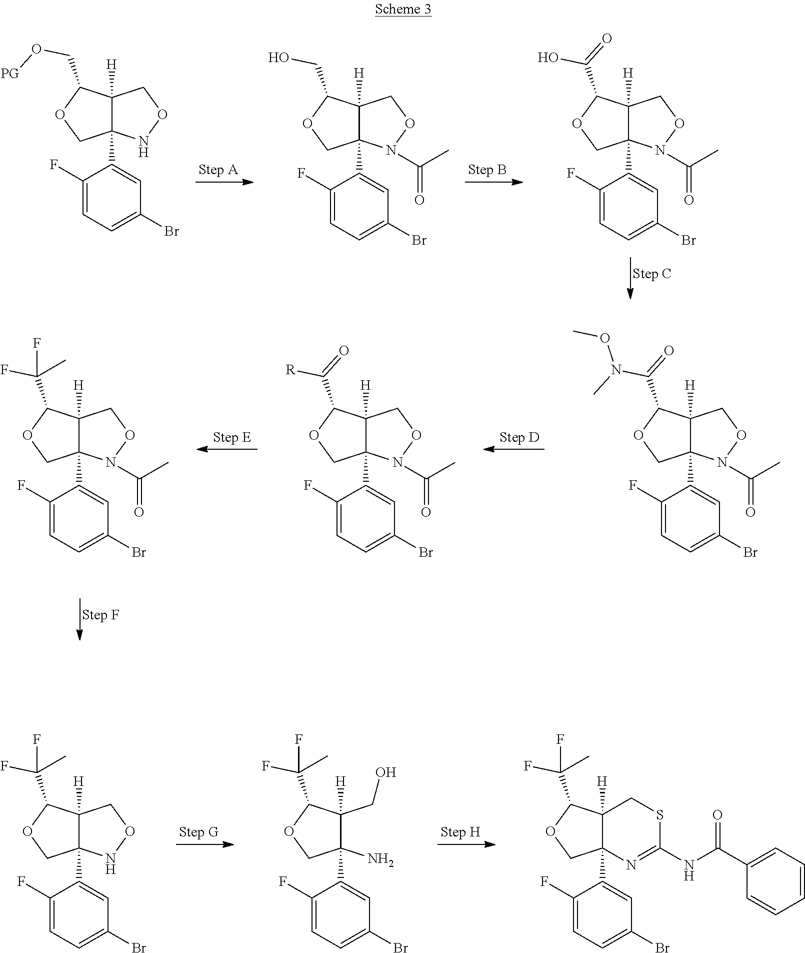

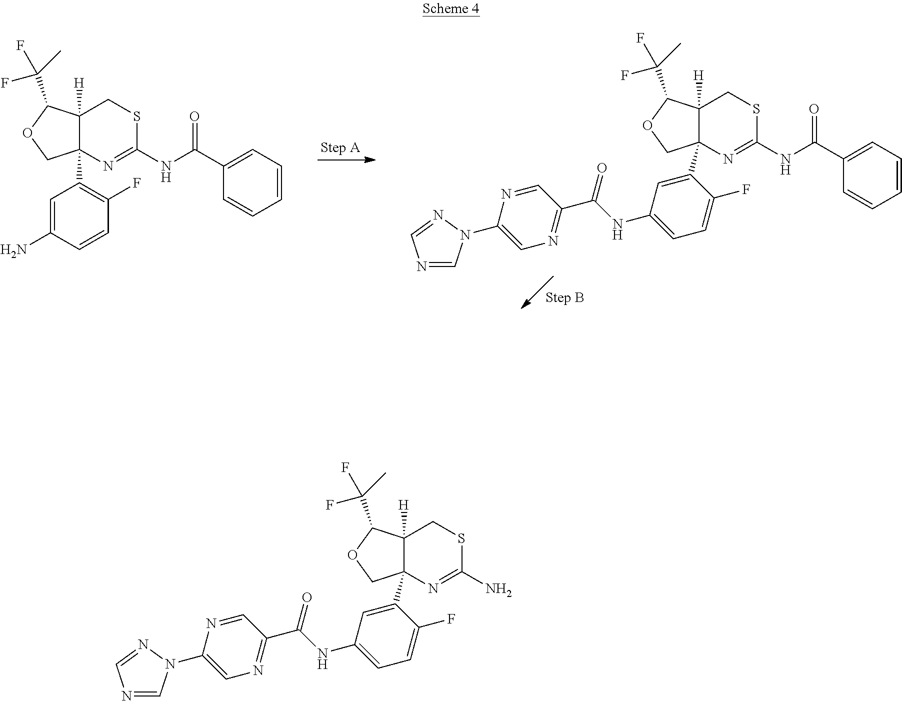 [SMILES: CC(F)(F)[C@H]1OC[C@]2(c3cc(N)ccc3F)N=C(NC(=O)c3ccccc3)SC[C@H]12 ; CC(F)(F)[C@H]1OC[C@]2(c3cc(NC(=O)c4cnc(-n5cncn5)cn4)ccc3F)N=C(NC(=O)c3ccccc3)SC[C@H]12 ; CC(F)(F)[C@H]1OC[C@]2(c3cc(NC(=O)c4cnc(-n5cncn5)cn4)ccc3F)N=C(N)SC[C@H]12]

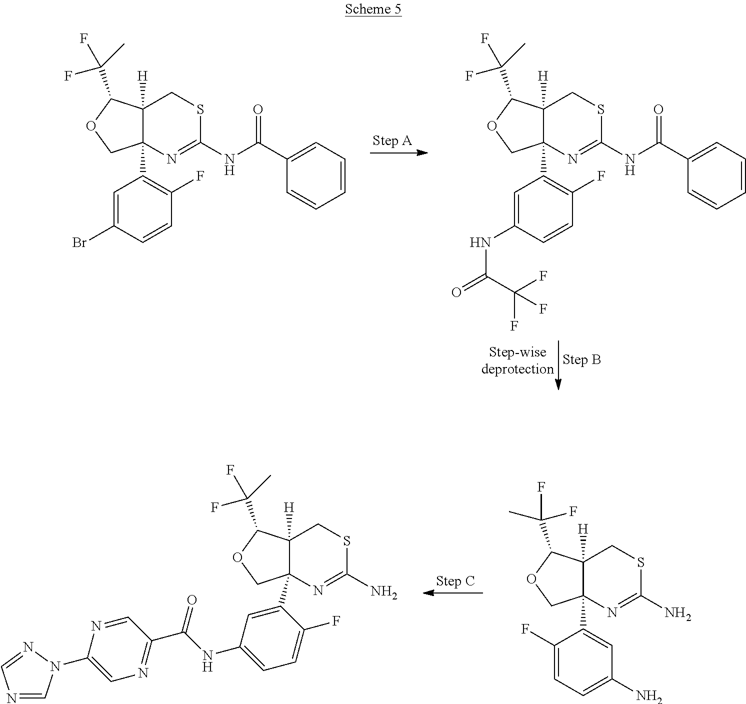 [SMILES: CC(F)(F)[C@H]1OC[C@]2(c3cc(Br)ccc3F)N=C(NC(=O)c3ccccc3)SC[C@H]12 ; CC(F)(F)[C@H]1OC[C@]2(c3cc(NC(=O)C(F)(F)F)ccc3F)N=C(NC(=O)c3ccccc3)SC[C@H]12 ; CC(F)(F)[C@H]1OC[C@]2(c3cc(NC(=O)c4cnc(-n5cncn5)cn4)ccc3F)N=C(N)SC[C@H]12 ; CC(F)(F)[C@H]1OC[C@]2(c3cc(N)ccc3F)N=C(N)SC[C@H]12]

A pharmaceutically acceptable salt of the compounds of the invention, such as a hydrochloride salt, can be formed, for example, by reaction of an appropriate free base of Formula I, and an appropriate pharmaceutically acceptable acid such as hydrochloric acid, p-toluenesulfonic acid, or malonic acid in a suitable solvent such as diethyl ether under standard conditions well known in the art. Additionally, the formation of such salts can occur simultaneously upon deprotection of a nitrogen protecting group. The formation of such salts is well known and appreciated in the art. See, for example, Gould, P. L., “Salt selection for basic drugs,” International Journal of Pharmaceutics, 33: 201-217 (1986); Bastin, R. J., et al. “Salt Selection and Optimization Procedures for Pharmaceutical New Chemical Entities,”

The following preparations and examples further illustrate the invention.

### Preparation 1

### (2S)-1-Trityloxybut-3-en-2-ol

Scheme 1, step A: Stir trimethylsulfonium iodide (193.5 g, 948.2 mmol) in THF (1264 mL) at ambient temperature for 75 minutes. Cool mixture to −50° C. and add n-butyllithium (2.5 mol/L in hexanes, 379 mL, 948.2 mmol) via cannula, over a period of 30 minutes. Allow the reaction to gradually warm to −30° C. and stir for 60 minutes. Add (2S)-2-trityloxymethyl oxirane (100 g, 316.1 mmol) portion wise, keeping the temperature below −10° C. After the complete addition, allow the reaction mixture to warm to room temperature and stir for 2 hours. Pour the reaction into saturated ammonium chloride, separate the phases, and extract the aqueous phase with ethyl acetate. Combine the organic layers and dry over magnesium sulfate. Filter and concentrate under reduced pressure to give a residue. Purify the residue by silica gel chromatography, eluting with methyl t-butyl ether:hexanes (10-15% gradient), to give the title compound (56.22 g, 54%). ES/MS m/z 353 (M+Na).

### Alternate Preparation 1

### (2S)-1-Trityloxybut-3-en-2-ol

Scheme 2, step A starting material: Add triphenylmethyl chloride (287 g, 947.1 mmol), DMAP (7.71 g, 63.1 mmol) and triethylamine (140 g, 1383.5 mmol) to a solution of (2S)-but-2-ene-1,2-diol (prepared as in JACS, 1999, 121, 8649) (64.5 g, 631 mmol) in dichloromethane (850 mL). Stir for 24 hours at 24° C. Add 1 N aqueous citric acid (425 mL). Separate the layers and concentrate the organic extract under reduced pressure to dryness. Add methanol (900 mL) and cool to 5° C. for 1 hour. Collect the solids by filtration and wash with 5° C. methanol (50 mL). Discard the solids and concentrate the mother liquor under reduced pressure to dryness. Add toluene (800 mL) and concentrate to a mass of 268 g to obtain the title compound (129 g, 67%) in a 48 wt % solution of toluene.

### Preparation 2

### 1-Morpholino-2-[(1 S)-1-(trityloxymethyl)allyloxy]ethanone

Scheme 2, step A: Add tetrabutyl ammonium hydrogen sulfate (83.2 g, 245.0 mmol) and 4-(2-chloroacetyl)morpholine (638.50 g, 3902.7 mmol) to a solution of 1-trityloxybut-3-en-2-ol (832.4, 2519 mmol) in toluene (5800 mL) that is between 0 and 5° C. Add sodium hydroxide (1008.0 g, 25202 mmol) in water (1041 mL). Stir for 19 hours between 0 and 5° C. Add water (2500 mL) and toluene (2500 mL). Separate the layers and wash the organic extract with water (2×3500 mL). Concentrate the organic extract under reduced pressure to dryness. Add toluene (2500 mL) to the residue and then add n-heptane (7500 mL) slowly. Stir for 16 hours. Collect the resulting solids by filtration and wash with n-heptane (1200 mL). Dry the solid under vacuum to obtain the title compound (1075.7 g, 98%).

### Preparation 3

### 1-(5-Bromo-2-fluoro-phenyl)-2-[(1 S)-1-(trityloxymethyl)allyloxy]ethanone

Scheme 2, step B: Add a 1.3 M solution of isopropyl magnesium chloride lithium chloride complex (3079 mL, 2000 mmol) in THF to a solution of 4-bromo-1-fluoro-2-iodobenze (673.2 g, 2237.5 mmol) in toluene (2500 mL) at a rate to maintain the reaction temperature below 5° C. Stir for 1 hour. Add the resulting Grignard solution (5150 mL) to a solution of 1-morpholino-2-[(1S)-1-(trityloxymethyl)allyloxy]ethanone (500 g, 1093 mmol) in toluene (5000 mL) at a rate to maintain the reaction temperature below 5° C. Stir for 3 hours maintaining the temperature below 5° C. Add additional prepared Grignard solution (429 mL) and stir for 1 hour. Add a 1 N aqueous citric acid solution (5000 mL) at a rate to maintain the temperature below 5° C. Separate the layers and wash the organic extract with water (5000 mL). Concentrate the solution under reduced pressure to dryness. Add methanol (2000 mL) to the residue and concentrate to give the title compound as a residue (793 g, 73.4% potency, 83%).

### Preparation 4

### 1-(5-Bromo-2-fluoro-phenyl)-2-[(1S)-1-(trityloxymethyl)allyloxy]ethanone oxime

Scheme 2, step C: Add hydroxylamine hydrochloride (98.3 g) to 1-(5-bromo-2-fluoro-phenyl)-2-[(1S)-1-(trityloxymethyl)allyloxy]ethanone (450 g, 707 mmol) and sodium acetate (174 g) in methanol (3800 mL). Heat the solution to 50° C. for 2 hours. Cool to 24° C. and concentrate. Add water (1000 mL) and toluene (1500 mL) to the residue. Separate the layers and extract the aqueous phase with toluene (500 mL). Combine the organic extract and wash with water (2×400 mL). Concentrate the solution under reduced pressure to give the title compound as a residue (567 g, 61.4% potency, 88%).

### Preparation 5

### tert-Butyl 2-[(1 S)-1-(trityloxymethyl)allyloxy]acetate

Scheme 1, step B: Add (2S)-1-trityloxybut-3-en-2-ol (74.67 g, 226.0 mmol) to a solution of tetra-N-butylammonium sulfate (13.26 g, 22.6 mmol) in toluene (376 mL). Add sodium hydroxide (50% mass) in water (119 mL) followed by tert-butyl-2-bromoacetate (110.20 g, 565.0 mmol). Stir reaction mixture for 18 hours at ambient temperature. Pour into water, separate the phases, and extract the aqueous phase with ethyl acetate. Combine the organic layers and dry over magnesium sulfate. Filter the mixture and concentrate under reduced pressure to give the title compound (77.86 g, 77%). ES/MS m/z 467 (M+Na).

### Preparation 6

### (1E)-2-[(1S)-1-(Trityloxymethyl)allyloxy]acetaldehyde oxime

Scheme 1, step C: Cool a solution of tert-butyl 2-[(1S)-1-(trityloxymethyl)allyloxy]acetate (77.66 g, 174.7 mmol) in dichloromethane (582.2 mL) to −78° C. Add a solution of diisobutylaluminum hydride in hexanes (1 mol/L, 174.7 mL) dropwise over a period of 35 minutes and maintain the temperature below −70° C. Stir at −78° C. for 5 hours. Add hydrochloric acid in water (2 mol/L, 192.1 mL) to the reaction mixture dropwise, keeping the temperature below −60° C. Allow the reaction to gradually warm to ambient temperature and stir for 60 minutes. Separate the organic extract and wash with saturated sodium bicarbonate. Dry the solution over magnesium sulfate, filter, and concentrate under reduced pressure to give a residue. Dissolve the residue in dichloromethane. Add sodium acetate (28.66 g, 349.3 mmol), followed by hydroxylamine hydrochloride (18.21 g, 262.0 mmol). Stir at ambient temperature for 18 hours. Pour into water, separate the phases, and extract the aqueous phase with dichloromethane. Combine the organic layers and dry over magnesium sulfate. Filter the mixture and concentrate under reduced pressure to give the title compound (68.38 g, 101%). ES/MS m/z 386 (M−H).

### Preparation 7

### (3aR,4S)-4-(Trityloxymethyl)-3,3a,4,6-tetrahydrofuro[3,4-c]isoxazole

Scheme 1, step D: Cool a solution of (1E)-2-[(1S)-1-(trityloxymethyl)allyloxy]acetaldehyde oxime (55.57 g, 143.4 mmol) in tert-butyl methyl ether (717 mL) to 5° C. Add sodium hypochlorite (5% in water, 591 mL, 430.2 mmol) dropwise, keeping the temperature below 10° C. Stir at 10° C. for 30 minutes. Allow the reaction to warm to 15° C. Stir at 15° C. for 18 hours. Dilute the reaction mixture with ethyl acetate and wash with saturated sodium bicarbonate. Separated the phases, wash the organic phase with a 5% sodium hydrogen sulphite solution and brine. Dry the solution over magnesium sulfate, filter, and concentrate under reduced pressure to give a residue. Purify the residue by silica gel chromatography, eluting with 50% methyl tert-butyl ether/dichloromethane:hexanes (20-27% gradient), to give the title compound (35.84 g, 65%). ES/MS m/z 408 (M+Na).

### Preparation 8

### (3aR,4S,6aR)-6a-(5-Bromo-2-fluoro-phenyl)-4-(trityloxymethyl)-3,3a,4,6-tetrahydrofuro[3,4-c]isoxazole

Scheme 1, step E: Cool a solution of 4-bromo-1-fluoro-2-iodo-benzene (86.94 g, 288.9 mmol) in THF (144.5 mL) and toluene (1445 mL) to −78° C. Add n-butyllithium (2.5 M in hexanes, 120 mL, 288.9 mmol) dropwise, keeping the temperature below −70° C. Stir for 30 minutes at −78° C. Add boron trifluoride diethyl etherate (36.5 mL, 288.9 mmol) dropwise, keeping temperature below −70° C. Stir the solution for 30 minutes at −78° C. Add a solution of (3aR,4S)-4-(trityloxymethyl)-3,3a,4,6-tetrahydrofuro[3,4-c]isoxazole (55.69 g, 144.5 mmol) in THF (482 mL) dropwise to the reaction, over a period of 30 minutes, keeping temperature below −65° C. Stir at −78° C. for 90 minutes. Rapidly add saturated ammonium chloride, keeping temperature below −60° C. Pour into brine, and extract the aqueous phase with ethyl acetate. Combine the organic extract and dry over magnesium sulfate. Filter and concentrate under reduced pressure to give a residue. Purify the residue by silica gel chromatography, eluting with 10-15% diethyl ether:hexanes (0-70% gradient), to give the title compound (36.52 g, 45%). ES/MS m/e (⁷⁹Br/⁸¹Br) 560/562 [M+H].

### Alternate Preparation 8

Scheme 2, step D: Heat a solution of 1-(5-bromo-2-fluoro-phenyl)-2-[(1S)-1-(trityloxymethyl)allyloxy]ethanone oxime (458 g, 502 mmol) and hydroquinone (56.3 g 511 mmol) in toluene (4000 mL) to reflux under nitrogen for 27 hours. Cool the solution to 24° C. and add aqueous sodium carbonate (800 mL). Separate the layers and extract the aqueous phase with toluene (300 mL). Combine the organic extract and wash with water (2×500 mL). Concentrate the solution under reduced pressure to give a residue. Add isopropyl alcohol (1500 mL) and heat to reflux. Cool to 24° C. and collect the solids by filtration. Dry the solid under vacuum to obtain the title compound (212 g, 75%).

### Preparation 9

### 1-[(3 aR,4S,6aS)-6a-(5-Bromo-2-fluoro-phenyl)-4-(trityloxymethyl)-3,3a,4,6-tetrahydrofuro[3,4-c]isoxazol-1-yl]ethanone

Scheme 2, step E: Add acetyl chloride (35.56 g, 503.9 mmol) to a solution of (3aR,4S,6aR)-6a-(5-bromo-2-fluoro-phenyl)-4-(trityloxymethyl)-3,3a,4,6-tetrahydrofuro[3,4-c]isoxazole (235.3 g, 420 mmol), DMAP (5.13 g, 42.0 mmol), and pyridine (66.45 g, 840.1 mmol) in dichloromethane (720 mL) under nitrogen, maintaining internal temperature below 5° C. Stir for 1 hour and then add water (300 mL) and 1 M sulfuric acid (300 mL). Stir the mixture for 10 minutes and allow the layers to separate. Collect the organic extract and wash with saturated sodium carbonate (500 mL) and water (500 mL). Dry the solution over magnesium sulfate. Filter and concentrate under reduced pressure to give 1-[(3aR,4S,6aS)-6a-(5-Bromo-2-fluoro-phenyl)-4-(trityloxymethyl)-3,3a,4,6-tetrahydrofuro[3,4-c]isoxazol-1-yl]ethanone (235 g, 93%) as a grey solid.

### Preparation 10

### 1-[(3aR,4S,6aS)-6a-(5-Bromo-2-fluorophenyl)-4-(hydroxymethyl)tetrahydro-1H,3H-furo[3,4-c][1,2]oxazol-1-yl]ethanone

Scheme 3, step A: In a 20 L jacketed reactor add acetyl chloride (290 mL, 4075 mmol) to a solution of (3aR,4S,6aR)-6a-(5-bromo-2-fluoro-phenyl)-4-(trityloxymethyl)-3,3a,4,6-tetrahydrofuro[3,4-c]isoxazole (1996 g, 3384 mmol), DMAP (56.0 g, 458 mmol), pyridine (500 mL, 6180 mmol) in dichloromethane (10 L) under nitrogen maintaining internal temperature below 10° C. After complete addition (1 hour) warm to 20° C. and stir overnight. If reaction is incomplete, add acetyl chloride, DMAP, pyridine, and dichloromethane until complete reaction is observed. Cool the reaction mixture to 0° C. and slowly add water (5 L), stir the reaction mixture at 10° C. for 30 minutes and allow the layers to separate. Collect the organic extract and wash the aqueous with dichloromethane (1 L). Wash the combined organic extracts with 1 N aqueous hydrochloric acid (2×4 L), extract the aqueous with dichloromethane (2×1 L). Wash the combined organic extracts with water (4 L) and remove the solvent under reduced pressure give total volume of approximately 5 L. Add 90% formic acid (1800 mL) and stand at ambient temperature for 3 days. Warm to 40° C. for 2 hours then remove the solvent under reduced pressure. Dilute the residue with methanol (4 L) and slowly add saturated aqueous sodium carbonate (3 L). Add solid sodium carbonate (375 g) to adjust the pH to 8-9. Stir at 45° C. for 1 hour then cool to ambient temperature. Remove the solids by filtration, washing with methanol (4×500 mL) then treat with 2 N aqueous sodium hydroxide (100 mL) and stand at ambient temperature for 1 hour. Remove the solids by filtration, washing with methanol (2×100 mL). Evaporate the solvent under reduced pressure and partition the residue between ethyl acetate (5 L) and water (2 L). Extract the aqueous with ethyl acetate (2 L) and wash the combined organic extracts with brine (2×1 L). Remove the solvent under reduced pressure, add methyl tert-butyl ether (2.5 L) and evaporate to dryness. Add methyl tert-butyl ether (4 L) and stir at 65° C. for 1 hour cool to ambient temperature and collect the solids by filtration, washing with methyl tert-butyl ether (3×500 mL). Dry under vacuum to a beige solid. Heat this solid in toluene (7.5 L) to 110° C. until fully dissolved, cool to 18° C. over 1 hour, and stir at this temperature for 1 hour. Warm to 40° C. and when precipitate forms, cool to 18° C. once more. Stir for 45 minutes then collect solids by filtration, washing with toluene (2×500 mL). Dry the solid under vacuum to obtain the title compound (443.1 g, 36%, 95% purity by LCMS). Evaporate the filtrate under vacuum to give a residue. Purify the residue by silica gel flash chromatography, eluting with 20% to 100% ethyl acetate in isohexane. Slurry the product containing fractions in methyl tert-butyl ether (2 L) at 60° C. for 30 minutes, cool to ambient temperature, and collect the solids by filtration, washing with methyl tert-butyl ether (2×200 mL). Dry the solids under vacuum to give the title compound as a beige crystalline solid (304 g, 24%, 88% purity by LCMS). Evaporate the filtrate under vacuum to a residue. Purify the residue by silica gel flash chromatography, eluting with 20% to 100% ethyl acetate in isohexane to give the title compound (57.8 g, 5%, 88% purity by LCMS). ES/MS: m/z (⁷⁹Br/⁸¹Br) 360.0/362.0 [M+H].

### Alternate Preparation 10

Scheme 3, step A: Add 1-[(3aR,4S,6aS)-6a-(5-bromo-2-fluoro-phenyl)-4-(trityloxymethyl)-3,3a,4,6-tetrahydrofuro[3,4-c]isoxazol-1-yl]ethanone (69 g, 114.5 mmol) to a 15° C. solution of p-toluenesulfonic acid monohydrate (2.2 g, 11.45 mmol), dichloromethane (280 mL) and methanol (700 mL). Stir for 18 hours and then remove the solvent under reduced pressure. Dilute the residue with dichloromethane (350 mL) and add 1 M aqueous sodium carbonate (140 mL) and water (140 mL). Separate the layers and evaporate the organic layer under reduced pressure. Add toluene (350 mL) to the residue and heat to reflux for 1 hour. Cool to 10-15° C. at a rate of 10° C./hour. Collect the solids by filtration and wash with toluene (70 mL). Dry the solid under vacuum to obtain the title compound (30 g, 65%) as a grey solid.

### Preparation 11

### (3aR,4S,6aS)-1-Acetyl-6a-(5-bromo-2-fluoro-phenyl)-3,3a,4,6-tetrahydrofuro[3,4-c]isoxazole-4-carboxylic Acid

Scheme 3, step B: Add water (2 L) to a suspension of 1-[(4S,6aS)-6a-(5-bromo-2-fluoro-phenyl)-4-(hydroxymethyl)-3,3a,4,6-tetrahydrofuro[3,4-c]isoxazol-1-yl]ethanone (804.9 g, 2177 mmol), TEMPO (40.0 g, 251 mmol) in acetonitrile (4.5 L) in a 20 L jacketed reactor and cool to an internal temperature of 5° C. Add (diacetoxyiodo)benzene (1693 g, 4993.43 mmol) portionwise over 30 minutes. Control the exotherm using reactor cooling and then hold at 20° C. until LCMS shows complete reaction. Slowly add a suspension of sodium bisulfite (70 g, 672.68 mmol) in water (300 mL) at ambient temperature, maintaining the internal temperature below 25° C. Stir for 30 minutes and then cool to 5° C. Add water (2 L), then slowly add 47 wt % aqueous sodium hydroxide (780 mL) over a period of 1 hour maintaining the internal temperature below 10° C. Add ethyl acetate (2 L) and isohexane (5 L), stir vigorously and separate the layers. Extract the biphasic organic layers with water (1 L) and wash the combined aqueous with methyl tert-butyl ether (2.5 L). Cool the aqueous extracts to 5° C. and slowly add 37% hydrochloric acid (1.4 L) over 30 minutes maintaining the internal temperature around 5° C. Add ethyl acetate (5 L), separate the layers and wash the organic with brine (3×1 L). Extract the combined aqueous extracts with ethyl acetate (2.5 L), wash the combined organics with brine (1 L), then dry with sodium sulfate, and filter. Dilute the organics with heptane (2.5 L) and evaporate to dryness under reduced pressure. Add methyl tert-butyl ether (1.5 L) and heptane (1.5 L) and evaporate to dryness. Add heptane (2.5 L) and evaporate to dryness twice. Add heptane (500 mL) and methyl tert-butyl ether (500 mL) and stir at 40° C. for 30 minutes then collect the precipitate by filtration, washing with heptane/methyl tert-butyl ether (1:1, 1 L) then methyl tert-butyl ether (3×300 mL) and air dry to give the title compound as a beige crystalline solid (779 g, 91%). ES/MS: m/z (⁷⁹Br/⁸¹Br) 374.0/376.0 [M+H].

[α]_{D}²⁰=−19.0° (C=1.004, chloroform).

### Alternate Preparation 11

Scheme 3, step B: Add water (150 mL) and acetonitrile (150 mL) to 1-[(4S,6aS)-6a-(5-bromo-2-fluoro-phenyl)-4-(hydroxymethyl)-3,3a,4,6-tetrahydrofuro[3,4-c]isoxazol-1-yl]ethanone (30 g, 73.3 mmol), TEMPO (1.14 g, 7.30 mmol) and (diacetoxyiodo) benzene (51.9 g, 161 mmol). Cool to 15° C. and stir for 2 hours. Slowly add sodium thiosulfate (21 g) and potassium carbonate (22 g) in water (150 mL) at ambient temperature. Stir for 1 hour and then add methyl tert-butyl ether (150 mL). Separate the layers and adjust the pH of the aqueous layer to 2-3 with concentrated sulfuric acid. Add ethyl acetate (150 mL) and separate the layers. Evaporate the organic layer to dryness under reduced pressure. Add n-heptane (90 mL) and heat to reflux for 1 hour. Cool to 15° C. and then collect the precipitate by filtration, washing with n-heptane (90 mL). Dry under vacuum to give the title compound as a white solid (27 g, 98%).

### Preparation 12

### (3aR,4S,6aS)-1-Acetyl-6a-(5-bromo-2-fluorophenyl)-N-methoxy-N-methyltetrahydro-1H,3H-furo[3,4-c][1,2]oxazole-4-carboxamide

Scheme 3, step C: In a 10 L jacketed reactor, cool a solution of (3aR,4S,6aS)-1-acetyl-6a-(5-bromo-2-fluoro-phenyl)-3,3a,4,6-tetrahydrofuro[3,4-c]isoxazole-4-carboxylic acid (771 g, 2019 mmol) in dichloromethane (7.0 L) to 0° C. under nitrogen and add CDI (400 g, 2421 mmol) portionwise over 40 minutes. Cool the reactor jacket to −20° C. and stir for 1 hour and then add N,O-dimethylhydroxylamine hydrochloride (260.0 g, 2612 mmol) portionwise over about 30 minutes. Stir at −20° C. for 1 hour, at 0° C. for 2 hours, and at 10° C. for 7 hours. Add CDI (175 g, 1058 mmol) and stir at 10° C. overnight. Add further CDI (180 g, 1088 mmol) at 10° C. and stirr for 1 hour then add N,O-dimethylhydroxylamine hydrochloride (140 g, 1407 mmol) and continue stirring at 10° C. If the reaction is incomplete, further charges of CDI followed by N,O-dimethylhydroxylamine hydrochloride can be made until complete reaction is observed. Cool the reaction mixture to 5° C. and wash with 1 N aqueous hydrochloric acid (5 L) then 2 N aqueous hydrochloric acid (5 L). Extract the combined aqueous solution with dichloromethane (1 L), combine the organic extract and wash with water (2.5 L), 1 N aqueous sodium hydroxide (2.5 L), and water (2.5 L), dry over magnesium sulfate, filter, and evaporate under reduced pressure to give a residue. Add methyl tert-butyl ether (3 L) and evaporate under reduced pressure. Add further methyl tert-butyl ether (2 L) and stir at 50° C. for 1 hour, cool to 25° C. and stir for 30 minutes. Collect the resulting solids by filtration, wash with methyl tert-butyl ether (2×500 mL) and dry under vacuum to give the title compound (760 g, 88%) as a white solid. ES/MS: m/z (⁷⁹Br/⁸¹Br) 417.0/419.0 [M+H].

### Alternate Preparation 12

Scheme 3, step C: Cool a solution of (3aR,4S,6aS)-1-acetyl-6a-(5-bromo-2-fluoro-phenyl)-3,3a,4,6-tetrahydrofuro[3,4-c]isoxazole-4-carboxylic acid (27 g, 70.7 mmol) in N,N-dimethylformamide (135 mL) to 0° C. under nitrogen and add CDI (14.9 g, 91.9 mmol). Stir for 1 hour and then add N,O-dimethylhydroxylamine hydrochloride (9.0 g, 92 mmol) and triethylamine (14.3 g, 141 mmol). Stir at 15° C. for 16 hours. Cool the reaction mixture to 0° C. and add 0.5 M aqueous sulfuric acid (675 mL). Stir for 1 hour. Collect the resulting solids by filtration. Slurry the solids in methyl tert-butyl ether (90 mL) for 1 hour. Collect the solids by filtration, wash with methyl tert-butyl ether (30 mL). Dry under vacuum to give the title compound (23 g, 78%) as a solid.

### Preparation 13

### 1-[(3aR,4S,6aS)-1-Acetyl-6a-(5-bromo-2-fluoro-phenyl)-3,3a,4,6-tetrahydrofuro[3,4-c]isoxazol-4-yl]ethanone

Scheme 3, step D: In a 20 L jacketed reactor, cool a solution of (3aR,4S,6aS)-1-acetyl-6a-(5-bromo-2-fluorophenyl)-N-methoxy-N-methyltetrahydro-1H,3H-furo[3,4-c][1,2]oxazole-4-carboxamide (654.0 g, 1536 mmol) in THF (10 L) to −60° C. and add a 3.2 M solution of methylmagnesium bromide in 2-methyltetrahydrofuran (660 mL, 2110 mmol) dropwise, while maintaining the internal temperature below −40° C. Stir the reaction mixture at −40° C. for 30 minutes then cool to −50° C. and add a solution of 1 N aqueous hydrochloric acid (2 L) in THF (2 L) maintaining the internal temperature below −38° C. Increase the temperature to 10° C. and add ethyl acetate (5 L) and water (1 L), stir and allow internal temperature to reach 5° C. and separate the layers. Extract the aqueous layer with ethyl acetate (1 L) and combine the organic extracts. Wash the organic extracts with water (2 L) and extract the aqueous layer with ethyl acetate (1 L). Combine the organic extract and wash with brine (3×2 L) then dry over magnesium sulfate, filter, and evaporate under reduced pressure to a residue. Add cyclohexane (2.5 L), stir at 60° C. for 1 hour then at 20° C. for 30 minutes, and collect the solid by filtration, washing with cyclohexane (500 mL). Dry the solid under vacuum to obtain the title compound as a white solid (565 g, 99%). ES/MS: m/z (⁷⁹Br/⁸¹Br) 372.0/374.0 [M+H], [α]_{D}²⁰=−58.0° (C=1.000, chloroform).

### Alternate Preparation 13

Scheme 3, step D: Cool a solution of (3aR,4S,6aS)-1-acetyl-6a-(5-bromo-2-fluorophenyl)-N-methoxy-N-methyltetrahydro-1H,3H-furo[3,4-c][1,2]oxazole-4-carboxamide (4.0 g, 9.59 mmol) in THF (60 mL) to −5° C. and add a 3.0 M solution of methylmagnesium bromide in 2-methyltetrahydrofuran (5.0 mL, 15 mmol) dropwise, while maintaining the internal temperature between −5 and 0° C. Stir the reaction mixture between −5 and 0° C. for 60 minutes then add a solution of saturated ammonium chloride (20 mL). Add methyl tert-butyl ether (40 mL), allow the internal temperature to reach 5° C. and separate the layers. Evaporate the organic layer under reduced pressure to a residue. Add n-heptane (50 mL), stir, and collect the solid by filtration. Dry the solid under vacuum to obtain the title compound as a solid (3.0 g, 77%).

### Preparation 14

### 1-[(3aR,4S,6aS)-6a-(5-Bromo-2-fluorophenyl)-4-(1,1-difluoroethyl)tetrahydro-1H,3H-furo[3,4-c][1,2]oxazol-1-yl]ethanone

Scheme 3, step E: Add 1-[(3aR,4S,6aS)-1-acetyl-6a-(5-bromo-2-fluoro-phenyl)-3,3a,4,6-tetrahydrofuro[3,4-c]isoxazol-4-yl]ethanone (5.08 g, 13.6 mmol) in a single portion to a stirred suspension of XtalFluor-M® (10.02 g, 39.18 mmol) in anhydrous dichloromethane (100 mL) at 0-5° C. Stir the mixture for 10 minutes and add triethylamine trihydrofluoride (4.5 mL, 27 mmol) dropwise over 10 minutes. Stir the reaction mixture in the ice-bath for 8 hours then warm to ambient temperature and stir overnight. Add saturated aqueous sodium carbonate (100 mL) and stir for 1 hour. Separate the layers and extract the aqueous with dichloromethane (2×50 mL). Combine the organic extracts and wash with saturated aqueous sodium bicarbonate (100 mL), 2 N aqueous hydrochloric acid (2×100 mL), and brine (100 mL). Evaporate to dryness to a light brown solid and dissolve in methyl tert-butyl ether (300 mL) at 60° C. Filter the hot solution and evaporate the filtrate to give a brown solid (5.3 g, 81%, 82% purity by LCMS) that is used without further purification. ES/MS: m/z (⁷⁹Br/⁸¹Br) 393.8/395.8 [M+H].

### Alternate Preparation 14

Scheme 3, step E: Add XtalFluor-M® (1.21 kg, 4.73 mol) in portions to a stirred solution of 1-[(3aR,4S,6aS)-1-acetyl-6a-(5-bromo-2-fluoro-phenyl)-3,3a,4,6-tetrahydrofuro[3,4-c]isoxazol-4-yl]ethanone (565 g, 1.51 mol) in anhydrous dichloromethane (5 L) at −14° C. Stir the mixture for 10 minutes and add triethylamine trihydrofluoride (550 g, 3.34 mol) dropwise over 20 minutes. Stir the reaction mixture at −10° C. for approximately 10 hours then warm to ambient temperature and stir overnight. Add 50% aqueous sodium hydroxide (750 mL) slowly, maintaining the internal temperature below 10° C., then add water (1.5 L) and saturated aqueous sodium hydrogen carbonate (1 L) and stir for 30 minutes. Separate the layers and extract the aqueous with dichloromethane (1 L). Combine the organic extracts and wash with brine (3 L), 2 N aqueous hydrochloric acid (5 L), and brine (3 L). Evaporate to give a residue and purify by silica gel chromatography eluting with 50-100% dichloromethane in iso-hexane then 10% methyl tert-butyl ether in dichloromethane to give the title compound as a white powder (467 g, 73%, 94% purity by LCMS). ES/MS: m/z (⁷⁹Br/⁸¹Br) 393.8/395.8 [M+H].

### Preparation 15

### (3aR,4S,6aS)-6a-(5-Bromo-2-fluoro-phenyl)-4-(1,1-difluoroethyl)-3,3a,4,6-tetrahydro-1H-furo[3,4-c]isoxazole

Scheme 3, step F: Add 37 wt % aqueous hydrochloric acid (1.3 L, 16 mol) to a solution of 1-[(3aR,4S,6aS)-6a-(5-bromo-2-fluorophenyl)-4-(1,1-difluoroethyl)tetrahydro-1H,3H-furo[3,4-c][1,2]oxazol-1-yl]ethanone (570 g, 1.45 mol) in 1,4-dioxane (5 L) in a 10 L jacketed reactor and stir at 100° C. for approximately 3 hours or until LCMS shows complete reaction. Cool the reaction mixture to 10° C., dilute with water (1 L) and add a mixture 50 wt % aqueous sodium hydroxide solution (800 mL) and water (1 L) slowly, maintaining the internal temperature below 20° C. Add ethyl acetate (2.5 L) and stir vigorously, before separating the layers and washing the organic phase with brine (2 L), further brine (1 L), and water (1 L). Dry over magnesium sulfate, filter, and concentrate to dryness under reduced pressure to give a residue. Add cyclohexane (2.5 L) and evaporate to dryness then repeat to obtain the title compound as a brown oil (527 g, 89%, 86% purity by LCMS). ES/MS: m/z (⁷⁹Br/⁸¹Br) 351.8/353.8 [M+H].

### Preparation 16

### [(2S,3R,4S)-4-Amino-4-(5-bromo-2-fluorophenyl)-2-(1,1-difluoroethyl)tetrahydrofuran-3-yl]methanol

Scheme 3, step G: Add zinc powder (6.0 g, 92 mmol) to a solution of (3aR,4S,6aS)-6a-(5-bromo-2-fluoro-phenyl)-4-(1,1-difluoroethyl)-3,3a,4,6-tetrahydro-1H-furo[3,4-c]isoxazole (5.06 g, 13.4 mmol) in acetic acid (100 mL) at ambient temperature and stir overnight. Dilute the mixture with ethyl acetate (200 mL) and water (300 mL) and stir vigorously while adding sodium carbonate (97 g, 915 mmol). Separate the layers and wash the organic layer with brine (2×200 mL), dry over magnesium sulfate, filter, and concentrate to give a residue. Purify the residue by silica gel chromatography eluting with 0% to 100% methyl tert-butyl ether in isohexane to give the title compound as a waxy solid (4.67 g, 89%, 90% purity by LCMS). ES/MS: m/z (⁷⁹Br/⁸¹Br) 354.0/356.0 [M+H].

### Alternate Preparation 16

Scheme 3, step G: Add zinc powder (200 g, 3.06 mol) portionwise to a solution of (3 aR,4S,6aS)-6a-(5-bromo-2-fluoro-phenyl)-4-(1,1-difluoroethyl)-3,3a,4,6-tetrahydro-1H-furo[3,4-c]isoxazole (304 g, 75% purity, 647 mmol) in acetic acid (2 L) and water (2 L) at 20° C. then warm to 40° C. and stir overnight. Dilute the mixture water (2 L) and stir vigorously while adding sodium carbonate (4 kg, 43.4 mol) then adjust to pH 8-9 with further sodium carbonate. Add ethyl acetate (5 L) and water (2.5 L), stir for 30 minutes and filter through diatomaceous earth washing with 2:1 acetonitrile/water. Separate the layers, extract the aqueous with ethyl acetate (2×2.5 L) and wash the combined organic extracts with brine (2×2.5 L), dry over magnesium sulfate, filter, and concentrate to give a residue. Purify the residue by SFC, column: Chiralpak AD-H (5), 50×250 mm; eluent: 12% ethanol (0.2% diethylmethylamine in CO₂; flow rate: 340 g/minute at UV 220 nm to give the title compound as a white solid (197.7 g, 84%). [α]_{D}²⁰=−6.93° (C=0.678, chloroform). ES/MS: m/z (⁷⁹Br/⁸¹Br) 354.0/356.0 [M+H].

### Preparation 17

### [(2S,3R,4S)-4-Amino-4-(5-bromo-2-fluoro-phenyl)-2-(trityloxymethyl)tetrahydrofuran-3-yl]methanol

Scheme 1, step F: Add (3aR,4S,6aR)-6a-(5-bromo-2-fluoro-phenyl)-4-(trityloxymethyl)-3,3a,4,6-tetrahydrofuro[3,4-c]isoxazole (31.30 g, 55.9 mmol) to acetic acid (186 mL) to give a suspension. Add zinc (25.6 g, 391 mmol) and stir the reaction mixture vigorously for 18 hours. Dilute the mixture with toluene and filter through diatomaceous earth. Concentrate the filtrate under reduced pressure. Solubilize the residue with ethyl acetate, wash with brine, and saturated sodium bicarbonate. Separate the phases, dry over magnesium sulfate, filter, and concentrate under reduced pressure to give the title compound (31.35 g, 99%). ES/MS m/e (⁷⁹Br/⁸¹Br) 562/564 [M+H].

### Preparation 18

### N-[[(3S,4R,5S)-3-(5-Bromo-2-fluoro-phenyl)-4-(hydroxymethyl)-5-(trityloxymethyl)tetrahydrofuran-3-yl]carbamothioyl]benzamide

Scheme 1, step G: Dissolve [(2S,3R,4S)-4-amino-4-(5-bromo-2-fluoro-phenyl)-2-(trityloxymethyl)tetrahydrofuran-3-yl]methanol (31.35 g, 55.73 mmol) in dichloromethane (557 mL) and cool to 5° C. Add benzoyl isothiocyanate (9.74 mL, 72.45 mmol). After addition is complete, allow the reaction mixture to warm to room temperature and stir for 2 hours. Pour into saturated sodium bicarbonate, separate the phases, and extract the aqueous phase with dichloromethane. Combine the organic extract and dry over magnesium sulfate. Filter the solution and concentrate under reduced pressure to give the title compound (42.95 g, 106%). ES/MS m/e (⁷⁹Br/⁸¹Br) 747/749 [M+Na].

### Preparation 19

### N-[(4aS,5S,7aS)-7a-(5-Bromo-2-fluorophenyl)-5-(1,1-difluoroethyl)-4a,5,7,7a-tetrahydro-4H-furo[3,4-d][1,3]thiazin-2-yl]benzamide

Scheme 3, step H: Add benzoyl isothiocyanate (1.80 mL, 13.3 mmol,) to a solution of [(2S,3R,4S)-4-amino-4-(5-bromo-2-fluorophenyl)-2-(1,1-difluoroethyl)tetrahydrofuran-3-yl]methanol (4.67 g, 11.9 mmol) in dichloromethane (20 mL) at ambient temperature for 1 hour until LCMS shows reaction is complete. Evaporate the reaction mixture to a residue under vacuum. Add cyclohexane (50 mL), warm to 60° C. and add methyl tert-butyl ether until precipitate is fully dissolved (100 mL). Filter the hot solution, cool to room temperature and slowly evaporate under reduced pressure until formation of a white precipitate. Remove the solvent under reduced pressure and dissolve the residue in anhydrous dichloromethane (30 mL), add pyridine (2.4 mL, 30 mmol), and cool the solution to −25° C. Add trifluoromethanesulfonic anhydride (2.2 mL 13 mmol) dropwise over 30 minutes and allow to warm 0° C. over 1 hour. Wash the reaction mixture with water (25 mL), 2 N aqueous hydrochloric acid (25 mL), water (25 mL), aqueous saturated sodium bicarbonate (25 mL), and water (25 mL), dry over magnesium sulfate, filter, and concentrated to dryness. Purify the residue by silica gel chromatography eluting with 5% methyl tert-butyl ether in dichloromethane to give the title compound as a light yellow foam (5.0 g, 76%, 90% purity by LCMS). ES/MS: m/z (⁷⁹Br/⁸¹Br) 499.0/501.0 [M+H].

### Alternate Preparation 19

Scheme 3, step H: Add benzoyl isothiocyanate (98 mL, 724.9 mmol,) to a solution of [(2S,3R,4S)-4-amino-4-(5-bromo-2-fluorophenyl)-2-(1,1-difluoroethyl)tetrahydrofuran-3-yl]methanol (197.6 g, 546.7 mmol) in dichloromethane (1.2 L) at 30° C. for 1 hour. Add CDI (101 g, 610.4 mmol) and stir at ambient temperature for 3 hours. Further charges of CDI can be made to ensure complete consumption of the thiourea intermediate. Heat to 90° C. for 42 hours and cool the solution to ambient temperature. Dilute the reaction mixture with ethyl acetate (2 L) and add 2 N aqueous hydrochloric acid (2 L), stir, add brine (1 L) and separate the layers. Wash the organic layer with 2 N aqueous hydrochloric acid (0.5 L), brine (2×1 L) and aqueous saturated sodium bicarbonate (1 L). Dry over magnesium sulfate, filter, and concentrate to give a residue. Purify the residue by silica gel chromatography eluting with 0-100% ethyl acetate in iso-hexane to give the title compound as a light yellow solid (234 g, 83%). ES/MS: m/z (⁷⁹Br/⁸¹Br) 499.0/501.0 [M+H].

### Preparation 20

### N-[(4aS,5S,7aS)-7a-(5-Bromo-2-fluoro-phenyl)-5-(trityloxymethyl)-4,4a,5,7-tetrahydrofuro[3,4-d][1,3]thiazin-2-yl]benzamide

Scheme 1, step H: Dissolve N-[[(3S,4R,5S)-3-(5-bromo-2-fluoro-phenyl)-4-(hydroxymethyl)-5-(trityloxymethyl)tetrahydrofuran-3-yl]carbamothioyl]benzamide (42.95 g, 59.18 mmol) in dichloromethane (591 mL) and cool to −20° C. Add pyridine (12.0 mL, 148.0 mmol), followed by trifluoromethanesulfonic anhydride (10.97 mL, 65.10 mmol). Monitor the addition keeping the temperature below −20° C. Stir the reaction mixture at −20° C. for 30 minutes. Allow the reaction mixture to warm to room temperature. Pour into saturated ammonium chloride, separate the phases, and extract the aqueous phase with dichloromethane. Combine the organic extract and dry over magnesium sulfate. Filter the solution and concentrate under reduced pressure to give the title compound (45.24 g, 108%). ES/MS m/e (⁷⁹Br/⁸¹Br) 707/709 [M+H].

### Preparation 21

### N-[(4aS,5S,7aS)-7a-(5-Bromo-2-fluoro-phenyl)-5-(hydroxymethyl)-4,4a,5,7-tetrahydrofuro[3,4-d][1,3]thiazin-2-yl]benzamide

Scheme 1, step I: Dissolve N-[(4aS,5S,7aS)-7a-(5-bromo-2-fluoro-phenyl)-5-(trityloxymethyl)-4,4a,5,7-tetrahydrofuro[3,4-d][1,3]thiazin-2-yl]benzamide (45.24, 63.93 mmol) in formic acid (160 mL) and stir at ambient temperature for 1 hour. Add water (29 mL) over a period of 5 minutes. Stir for 50 minutes. Concentrate the mixture under reduced pressure to a residue. Dissolve the residue in methanol (639 mL), add triethylamine (26.7 mL, 191.8 mmol), and stir overnight at ambient temperature. Pour into brine, separate the phases, and extract the aqueous phase with chloroform. Combine the organic extract and dry over magnesium sulfate. Filter and concentrate under reduced pressure to give a residue. Purify the residue by silica gel chromatography, eluting with acetone:hexanes (25-38% gradient), to give the title compound (16.04 g, 54%). ES/MS m/e (⁷⁹Br/⁸¹Br) 465/467 [M+H].

### Preparation 22

### (4aS,5S,7aS)-2-Benzamido-7a-(5-bromo-2-fluoro-phenyl)-4,4a,5,7-tetrahydrofuro[3,4-d][1,3]thiazine-5-carboxylic Acid

Scheme 1, step J: Add N-[(4aS,5S,7aS)-7a-(5-bromo-2-fluoro-phenyl)-5-(hydroxymethyl)-4,4a,5,7-tetrahydrofuro[3,4-d][1,3]thiazin-2-yl]benzamide (16.04 g, 34.47 mmol) to DMSO (172 mL). Add 2-iodoxybenzoic acid (35.56 g, 120.70 mmol) and stir at ambient temperature for 3 hours. Dilute the reaction mixture with chloroform (300 mL) and pour into saturated ammonium chloride (400 mL). Separate the organic phase and dry over magnesium sulfate. Filter the solution and concentrate under reduced pressure to give a residue. Dissolve the residue in ethyl acetate (400 mL) and wash with saturated ammonium chloride (2×250 mL). Separate the organic phase, dry over magnesium sulfate, filter, and concentrate under reduced pressure to give a residue. Dissolve the residue in a dichloromethane:methanol mixture and add diethyl ether until a solid precipitates. Collect the solid by filtration and dry under reduced pressure to give the title compound (5.78 g, 35%). ES/MS m/e (⁷⁹Br/⁸¹Br) 479/481 [M+H].

### Preparation 23

### (4aS,5S,7aS)-2-Benzamido-7a-(5-bromo-2-fluoro-phenyl)-N-methoxy-N-methyl-4,4a,5,7-tetrahydrofuro[3,4-d][1,3]thiazine-5-carboxamide

Scheme 1, step K: Dissolve (4aS,5S,7aS)-2-benzamido-7a-(5-bromo-2-fluoro-phenyl)-4,4a,5,7-tetrahydrofuro[3,4-d][1,3]thiazine-5-carboxylic acid (5.78 g, 12.1 mmol) in dichloromethane (201 mL) and N,O-dimethylhydroxylamine hydrochloride (1.76 g, 18.1 mmol). Add triethylamine (5.29 mL, 36.2 mmol) followed by HATU (7.02 g, 18.1 mmol). Stir at ambient temperature for 3 days. Pour into saturated ammonium chloride, separate the phases, and extract the aqueous phase with ethyl acetate. Combine the organic extracts and dry over magnesium sulfate. Filter and concentrate under reduced pressure to give a residue. Purify the residue by silica gel chromatography, eluting with ethyl acetate:dichloromethane (0-50% gradient) to give the title compound (4.15 g, 66%). ES/MS m/e (⁷⁹Br/⁸¹Br) 522/524 [M+H].

### Preparation 24

### N-[(4aS,5S,7aS)-5-Acetyl-7a-(5-bromo-2-fluoro-phenyl)-4,4a,5,7-tetrahydrofuro[3,4-d][1,3]thiazin-2-yl]benzamide

Scheme 1, step L: Add dropwise to a −78° C. solution of (4aS,5S,7aS)-2-benzamido-7a-(5-bromo-2-fluoro-phenyl)-N-methoxy-N-methyl-4,4a,5,7-tetrahydrofuro[3,4-d][1,3]thiazine-5-carboxamide (1.51 g, 2.89 mmol) in THF (57.8 mL) methylmagnesium bromide (3.0 mol/L in diethyl ether, 4.8 mL, 14.5 mmol). Stir the reaction at −78° C. for 5 minutes and allow to gradually warm to ambient temperature. Stir for 30 minutes. Quench the reaction with methanol (4 mL), dilute with saturated ammonium chloride, and extract with ethyl acetate. Combine the organic extract and dry over sodium sulfate. Filter and concentrate under reduced pressure to give a residue. Purify the residue by silica gel chromatography, eluting with ethyl acetate:hexanes (0-100% gradient) to give the title compound (1.28 g, 93%). ES/MS m/e (⁷⁹Br/⁸¹Br) 477/479 [M+Na].

### Preparation 25

### N-[(4aS,5S,7aS)-7a-(5-Bromo-2-fluoro-phenyl)-5-(1,1-difluoroethyl)-4,4a,5,7-tetrahydrofuro[3,4-d][1,3]thiazin-2-yl]benzamide

Scheme 1, step M: Add together dichloromethane (34 mL), Deoxo-Fluor® (1.52 mL, 6.88 mmol), and boron trifluoride diethyl etherate (0.89 mL, 6.88 mmol). Stir at ambient temperature for 2 hours. Add N-[(4aS,5S,7aS)-5-acetyl-7a-(5-bromo-2-fluoro-phenyl)-4,4a,5,7-tetrahydrofuro[3,4-d][1,3]thiazin-2-yl]benzamide (0.821 g, 1.72 mmol) in one portion, followed by triethylamine trihydrofluoride (1.13 mL, 6.88 mmol). Stir at ambient temperature for 18 hours. Pour into saturated ammonium chloride, separate the phases, and extract the aqueous phase with ethyl acetate. Combine the organic extract and dry over magnesium sulfate. Filter and concentrate under reduced pressure to give a residue. Purify the residue by silica gel chromatography, eluting with dichloromethane:hexanes (80-100% gradient), to give the title compound (0.552 g, 64%). ES/MS m/e (⁷⁹Br/⁸¹Br) 499/501 [M+H].

### Preparation 26

### N-[(5S,7aS)-5-(1,1-Difluoroethyl)-7a-{2-fluoro-5-[(trifluoroacetyl)amino]phenyl}-4a,5,7,7a-tetrahydro-4H-furo[3,4-d][1,3]thiazin-2-yl]benzamide

Scheme 4, step A: Dissolve N-[(4aS,5S,7aS)-7a-(5-bromo-2-fluorophenyl)-5-(1,1-difluoroethyl)-4a,5,7,7a-tetrahydro-4H-furo[3,4-d][1,3]thiazin-2-yl]benzamide (234 g, 454.6 mmol) in 1,4-dioxane (2 L) and add 4 Å molecular sieves (37 g), 2,2,2-trifluoroacetamide (91 g, 780.9 mmol), finely ground potassium carbonate (114 g, 824.9 mmol), sodium iodide (117 g, 780.6 mmol), copper (I) iodide (17.5 g, 91.9 mmol) and racemic trans-N,N′-dimethyl-1,2-cyclohexane diamine (20 g, 140.6 mmol) under a stream of nitrogen. Purge the vessel with 3 vacuum nitrogen switches and heat to 123° C. for 18 hours. Cool to ambient temperature and filter the solution through diatomaceous earth, and wash with ethyl acetate. Add saturated aqueous ammonium chloride (2 L) and vigorously stir for 45 minutes. Separate the layers and wash the organic layer with saturated aqueous ammonium chloride (3×1 L), brine (300 mL), dry over magnesium sulfate, filter, and evaporate to give a residue. Purify the residue by silica gel chromatography eluting with 0-100% ethyl acetate in iso-hexane to give the title compound as a light yellow solid (297.9 g, 95%, 81% purity). ES/MS: m/z 532.0 [M+H].

### Preparation 27

### N-[(4aS,5S,7aS)-7a-(5-Amino-2-fluoro-phenyl)-5-(1,1-difluoroethyl)-4,4a,5,7-tetrahydrofuro[3,4-d][1,3]thiazin-2-yl]benzamide

Scheme 1, step N: Combine N-[(4aS,5S,7aS)-7a-(5-bromo-2-fluoro-phenyl)-5-(1,1-difluoroethyl)-4,4a,5,7-tetrahydrofuro[3,4-d][1,3]thiazin-2-yl]benzamide (0.372 g, 0.74 mmol) and (1R,2R)—N,N′-dimethyl-1,2-cyclohexanediamine (0.037 mL, 0.22 mmol) in ethanol (30 ml). Add sodium azide (0.194 g, 2.98 mmol), followed by sodium L-ascorbate (0.66 M solution, 0.50 ml, 0.33 mmol). Purge the top of the flask with nitrogen and add cupric sulfate (0.33 M solution, 0.68 ml, 0.22 mmol). Heat the reaction mixture to 80° C. and stir for 5 hours. Cool the reaction and add cold water. Extract the mixture with ethyl acetate. Combine the organic extract and dry over sodium sulfate. Filter and concentrate under reduced pressure to give a residue. Combine the residue with palladium (10 mass % on carbon, 0.35 g, 0.16 mmol) in ethanol (50 ml) and THF (10 ml). Purge the mixture with nitrogen and with hydrogen. Stir at ambient temperature under 50 psi of hydrogen for 1 hour. Filter off the catalyst and wash with ethyl acetate. Concentrate the solution under reduced pressure to give a residue. Purify the residue by silica gel chromatography, eluting with ethyl acetate:dichloromethane (0-20% gradient), to give the title compound (0.2184 g, 67%). ES/MS m/z 436 (M+H).

### Alternate Preparation 27

Scheme 4, step B: Add 7 N ammonia in methanol (600 mL, 4.2 mol) to a stirred suspension of N-[(5S,7aS)-5-(1,1-difluoroethyl)-7a-{2-fluoro-5-[(trifluoroacetyl)amino]phenyl}-4a,5,7,7a-tetrahydro-4H-furo[3,4-d][1,3]thiazin-2-yl]benzamide (250 g, 80% purity, 376.3 mmol) in methanol (200 mL) at room temperature and stir at ambient temperature for 18 hours. Evaporate to dryness to give the title compound as a brown gum (190 g, 375.2 mmol, 86% purity). ES/MS: m/z 436.0 [M+H].

### Preparation 28

### (4aS,5S,7aS)-7a-(5-Amino-2-fluorophenyl)-5-(1,1-difluoroethyl)-4a,5,7,7a-tetrahydro-4H-furo[3,4-d][1,3]thiazin-2-amine

Scheme 4, step B: Dissolve N-[(4aS,5S,7aS)-7a-(5-amino-2-fluoro-phenyl)-5-(1,1-difluoroethyl)-4,4a,5,7-tetrahydrofuro[3,4-d][1,3]thiazin-2-yl]benzamide (216.4 g, 88% purity, 435.9 mmol) in pyridine (400 mL), ethanol (100 mL) and THF (300 mL). Add O-methylhydroxylamine hydrochloride (190 g, 2275.0 mmol) and stir at ambient temperature for 18 hours. Dilute with 2-methyltetrahydrofuran (1 L) and wash with water (2×300 mL). Isolate the organic layer and add 35% aqueous ammonium hydroxide (100 mL) to the aqueous. Extract with 2-methyltetrahydrofuran (300 mL) then saturate with sodium chloride and extract with 2-methyltetrahydrofuran (2×300 mL). Combine the organic extracts, wash with brine (300 mL) and evaporate to a residue. Dissolve in methanol (200 mL), add 7 N ammonia in methanol (100 mL, 700 mmol) and stir at room temperature for 18 hours. Further ammonia can be added if any trifluoracetamide impurity remains. Remove the solvent under reduced pressure and dissolve the residue in aqueous 2 N aqueous hydrochloric acid (1.5 L). Extract with dichloromethane (6×500 mL), combine the organic layers and remove the solvent under reduced pressure to a total volume of about 1 L. Wash with 2 N aqueous hydrochloric acid (300 mL) and combine all aqueous washings. Add 2-methyltetrahydrofuran (1 L) and stir vigorously while adjusting the pH to basic with sodium bicarbonate until no gas evolution is observed. Separate the layers and extract the aqueous with 2-methyltetrahydrofuran (2×500 mL). Dry the combined organic extracts with magnesium sulfate, filter, and evaporate to give a brown solid. Purify the residue by silica gel chromatography eluting with 0-100% dichloromethane in THF. Evaporate the product containing fractions with ethyl acetate/heptane to give the title compound as a fine beige powder (106 g, 70%, 95% purity). ES/MS: m/z 332.0 [M+H], [α]_{D}²⁰=+42.11° (C=0.532, chloroform).

### Preparation 29

### 5-(1H-1,2,4-Triazol-1-yl)pyrazine-2-carboxylic Acid

Stir a mixture of methyl 5-chloropyrazine-2-carboxylate (124 g, 718.55 mmol), 1H-1,2,4-triazole (198.5 g, 2874.2 mmol) and potassium carbonate (297.92 g, 2155.6 mmol) in N,N-dimethylformamide (1 L) at 100° C. for 15 hours. Cool to ambient temperature and pour into water (2 L). Adjust the pH of the solution to 2-3 using concentrated aqueous hydrochloric acid (about 500 mL) and stir for 30 minutes. Collect the resulting solid by filtration and wash with water. Add water (500 mL) and ethanol (500 mL), heat to 50-60° C. for 4 hours, and cool to ambient temperature. Collect the solids by filtration and dry under vacuum at 40° C. to give the title compound as a white solid. ES/MS: m/z 190.0 (M−H).

### Preparation 30

### N-[3-[(4aS,5S,7aS)-2-Benzamido-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide

Scheme 3, step A: Add together N-[(4as,5s,7as)-7a-(5-amino-2-fluoro-phenyl)-5-(1,1-difluoroethyl)-4,4a,5,7-tetrahydrofuro[3,4-d][1,3]thiazin-2-yl]benzamide (0.139 g, 0.32 mmol), 5-(1H-1,2,4-triazol-1-yl)pyrazine-2-carboxylic acid (0.0852 g, 0.45 mmol), and HOAt (0.0575 g, 0.41 mmol) in dichloromethane (4 ml): dimethylformamide (1 mL). Add N,N-diisopropylethylamine (0.11 mL, 0.63 mmol) to the solution followed by EDCI (0.079 g, 0.41 mmol) in one portion. Stir the reaction mixture at ambient temperature for 18 hours. Dilute the solution with ethyl acetate, wash with water and brine, and separate the phases. Extract with ethyl acetate. Combine the organic extract and dry over magnesium sulfate. Filter the solution and concentrate under reduced pressure to give a residue. Purify the residue by silica gel chromatography, eluting with ethyl acetate:dichloromethane (0-30% gradient), to give the title compound (0.1140 g, 59%). ES/MS m/z 609 (M+H).

### Example 1

### N-[3-[(4aS,5S,7aS)-2-Amino-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide

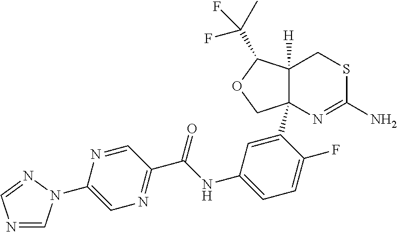 [SMILES: CC(F)(F)[C@H]1OC[C@]2(c3cc(NC(=O)c4cnc(-n5cncn5)cn4)ccc3F)N=C(N)SC[C@H]12]

Scheme 3, step B: Heat a mixture of N-[3-[(4aS,5S,7aS)-2-benzamido-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide (0.1148 g, 0.189 mmol), O-methylhydroxylamine hydrochloride (0.1575 g, 1.886 mmol), and pyridine (0.15 ml, 1.886 mmol) in THF (2 mL) and ethanol (2 mL) at 45° C. for 5 hours. Cool the reaction mixture to ambient temperature and stir for 2 days. Concentrate the solution under reduced pressure to give a residue. Purify the residue by silica gel chromatography, eluting with 7 N NH₃ in methanol:dichloromethane (0-³% gradient), to give the title compound (0.086 g, 90%). ES/MS m/z 505 (M+H).

### Alternative Preparation Example 1

Scheme 4 Step D: Stir (4aS,5S,7aS)-7a-(5-amino-2-fluorophenyl)-5-(1,1-difluoroethyl)-4a,5,7,7a-tetrahydro-4H-furo[3,4-d][1,3]thiazin-2-amine (96.5 g, 291 mmol) in ethyl acetate (1 L) under a nitrogen atmosphere at 50° C. and add 5-(1H-1,2,4-triazol-1-yl)pyrazine-2-carboxylic acid (84 g, 439.45 mmol) slowly to the warm solution. Stir for 10 minutes and add T3P® (1.67 M in ethyl acetate, 350 mL, 585 mmol) and stir at 50° C. for 17 hours. Cool to ambient temperature, dilute with dichloromethane (1 L) and stir while quenching with a solution of sodium carbonate in water (128 g, 1.21 mol in 1 L). Dilute with dichloromethane (1 L) and water (2 L) and stir vigorously for 1 hour. Filter through diatomaceous earth and wash with dichloromethane (3×500 mL), methanol (500 mL), water (500 mL), aqueous saturated sodium bicarbonate (500 mL), and 1:1 methanol:dichloromethane (6×500 mL). Separate the layers and extract the aqueous with dichloromethane (3×1 L). Combine all organic phases and evaporate to give a residue. Sonicate the residue in dichloromethane (1 L) for 15 minutes and collect the solids by filtration washing with dichloromethane (5×200 mL). Add saturated aqueous sodium hydrogen carbonate until pH 8 is obtained and stir vigorously with dichloromethane (1 L) and methanol (500 mL). Remove the solids by filtration and extract the filtrate with dichloromethane (2×500 mL). Dissolve the solids with dichloromethane:methanol (1:1, 500 mL) and combine this solution with the other organic phases. Remove the solvent under reduced pressure adding dichloromethane to maintain a solution and then once a final volume of about 300 mL is obtained, purify the solution by silica gel chromatography, eluting with 5% of 0.3 M ammonia/methanol in dichloromethane to give a light brown solid. Dissolve the solid in hot ethanol (2.5 L), filter while hot, and cool to ambient temperature over 1 hour. Collect the solids by filtration and wash with ethanol (2×250 mL) and dry under vacuum. Evaporate the filtrate to dryness and further purify by silica gel chromatography eluting first with 65% ethyl acetate in 50:1 iso-hexane/7 N ammonia in methanol then 50:1 ethyl acetate/7 N ammonia in methanol. If required, further purification can be completed by SFC, column: Chiralpak AD-H (5μ), 50×250 mm; eluent: 35% isopropanol (0.2% diethylmethylamine) in CO₂; flow rate: 300 g/minute at UV 220 nm. After evaporation and vacuum drying, slurry the material in ethanol (1.5 L) and stir with gentle warming between (36 and 45° C.) for 20 minutes. Collect the solid by filtration washing with ethanol (100 mL). Further material can be recovered from the filtrate; evaporate to dryness, reflux in ethanol, remove the solids by hot filtration and then cool the filtrate to ambient temperature. Collect solids by filtration, washing with ethanol and combine with the material obtained from the above filtration. Dry the combined solids under vacuum at 40° C. to give the title compound as a white solid (103.3 g, 68%, containing 2.5 wt % ethanol). ES/MS m/z 505.0 (M+H), [α]_{D}²⁰=+149.4° (C=1, chloroform).

### Example 1A

### N-[3-[(4aS,5S,7aS)-2-amino-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide 4-methylbenzenesulfonate

Dissolve N-[3-[(4aS,5S,7aS)-2-amino-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide (600 mg, 1.189 mmol) in acetone (9 mL) and water (1 mL). Heat the resulting suspension to 60° C. Add p-toluenesulfonic acid monohydrate (420 mg, 2.208 mmol) dissolved in acetone (1 mL). Stir the mixture overnight at 60° C. Cool the mixture to room temperature, filter the solids by vacuum and wash with acetone (1 mL) and air dry overnight to give the title compound (743 mg, 73%).

### X-Ray Powder Diffraction (XRD)

The XRD patterns of crystalline solids are obtained on a Bruker D4 Endeavor X-ray powder diffractometer, equipped with a CuKa source λ=1.54060 Å and a Vantec detector, operating at 35 kV and 50 mA. The sample is scanned between 4 and 40° in 20, with a step size of 0.0090 in 20, a scan rate of 0.5 seconds/step, with 0.6 mm divergence, 5.28 fixed anti-scatter, and 9.5 mm detector slits. The dry powder is packed on a quartz sample holder and a smooth surface is obtained using a glass slide. The crystal form diffraction patterns are collected at ambient temperature and relative humidity. It is well known in the crystallography art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g., The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature or humidity at which a sample is analyzed, sample displacement, or the presence or absence of an internal standard. In the present case, a peak position variability of 0.2 in 20 will take into account these potential variations without hindering the unequivocal identification of the indicated crystal form. Confirmation of a crystal form may be made based on any unique combination of distinguishing peaks (in units of ° 20), typically the more prominent peaks. The crystal form diffraction patterns, collected at ambient temperature and relative humidity, were adjusted based on NIST 675 standard peaks at 8.853 and 26.774 degrees 2-theta.

A prepared sample of crystalline N-[3-[(4aS,5S,7aS)-2-amino-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide 4-methylbenzenesulfonate is characterized by an XRD pattern using CuKa radiation as having diffraction peaks (2-theta values) as described in the Table below. Specifically, the pattern contains a peak at 17.30 in combination with one or more of the peaks selected from the group consisting of 14.8, 12.7, and 4.9; with a tolerance for the diffraction angles of 0.2 degrees.

TABLE 1
X-ray powder diffraction peaks of Example 1A.

| | Angle | Relative Intensity |
|---|---|---|
| Peak | (°2-Theta +/− 0.2°) | (% of most intense peak) |
| 1 | 4.9 | 48 |
| 2 | 9.4 | 14 |
| 3 | 12.7 | 52 |
| 4 | 14.8 | 60 |
| 5 | 17.3 | 100 |
| 6 | 19.8 | 44 |
| 7 | 24.9 | 35 |
| 8 | 25.3 | 37 |
| 9 | 26.8 | 19 |
| 10 | 28.2 | 14 |

### Example 1B

### N-[3-[(4aS,5S,7aS)-2-amino-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide Malonate

Add together N-[3-[(4aS,5S,7aS)-2-amino-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide (201 mg, 0.398 mmol) and malonic acid (104 mg, 0.999 mmol) in 95% ethanol-water (15 mL). Stir the mixture 65° C. until a solution a clear solution is obtained. A thick white solid precipitates after a few minutes. Stir the suspension for 1 hour at 55° C. and then cool to room temperature with stirring. Filter the solids under vacuum and air dry for 2 days to give the title compound (477 mg, 80%).

A prepared sample of crystalline N-[3-[(4aS,5S,7aS)-2-amino-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide malonate is characterized by an XRD pattern using CuKa radiation as having diffraction peaks (2-theta values) as described in Table 2 below. Specifically, the pattern contains a peak at 22.7 in combination with one or more of the peaks selected from the group consisting of 16.8, 17.2, and 24.0; with a tolerance for the diffraction angles of 0.2 degrees.

TABLE 2
X-ray powder diffraction peaks of Example 1B.

| | Angle | Relative Intensity |
|---|---|---|
| Peak | (°2-Theta +/− 0.2°) | (% of most intense peak) |
| 1 | 5.5 | 39 |
| 2 | 10.3 | 44 |
| 3 | 11.8 | 55 |
| 4 | 15.3 | 39 |
| 5 | 16.8 | 62 |
| 6 | 17.2 | 57 |
| 7 | 18.3 | 41 |
| 8 | 22.4 | 60 |
| 9 | 22.7 | 100 |
| 10 | 24.0 | 53 |

### Example 1C

### N-[3-[(4aS,5S,7aS)-2-amino-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide Hydrate

Suspend N-[3-[(4aS,5S,7aS)-2-amino-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide (116 mg, 0.23 mmol) in 1:1 THF:water (2 mL) at 70° C. Stir the solution for at least 2 days, filter the solid, and dry under a nitrogen stream to give the title compound.

A prepared sample of N-[3-[(4aS,5S,7aS)-2-amino-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide hydrateis characterized by an XRD pattern using CuKa radiation as having diffraction peaks (2-theta values) as described in Table 3 below. Specifically, the pattern contains a peak at 13.0 in combination with one or more of the peaks selected from the group consisting of 7.8, 10.5, 11.0, 14.9, 19.7, 21.3, and 26.9 with a tolerance for the diffraction angles of 0.2 degrees.

TABLE 3
X-ray powder diffraction peaks of Example 1C.

| | Angle | Relative Intensity |
|---|---|---|
| Peak | (°2-Theta +/− 0.2°) | (% of most intense peak) |
| 1 | 7.8 | 82 |
| 2 | 10.5 | 68 |
| 3 | 11.0 | 38 |
| 4 | 13.0 | 100 |
| 5 | 13.2 | 48 |
| 6 | 14.9 | 44 |
| 7 | 16.6 | 30 |
| 8 | 19.1 | 32 |
| 9 | 19.7 | 93 |
| 10 | 21.1 | 29 |
| 11 | 21.3 | 73 |
| 12 | 22.0 | 26 |
| 13 | 22.3 | 52 |
| 14 | 26.9 | 65 |

### In Vitro Assay Procedures:

To assess selectivity of BACE1 over BACE2, the test compound is evaluated in FRET-based enzymatic assays using specific substrates for BACE1 and BACE2 as described below. For in vitro enzymatic and cellular assays, the test compound is prepared in DMSO to make up a 10 mM stock solution. The stock solution is serially diluted in DMSO to obtain a ten-point dilution curve with final compound concentrations ranging from 10 μM to 0.05 nM in a 96-well round-bottom plate before conducting the in vitro enzymatic and whole cell assays.

### In Vitro Protease Inhibition Assays

### Expression of huBACE1:Fc and huBACE2:Fc

Human BACE1 (accession number: AF190725) and human BACE2 (accession number: AF 204944) are cloned from total brain cDNA by RT-PCR. The nucleotide sequences corresponding to amino acid sequences #1 to 460 are inserted into the cDNA encoding human IgG₁ (Fc) polypeptide (Vassar et al., Science, 286, 735-742 (1999)). This fusion protein of BACE1(1-460) or BACE2(1-460) and human Fc, named huBACE1:Fc and huBACE2:Fc respectively, are constructed into the pJB02 vector. Human BACE1(1-460):Fc (huBACE1:Fc) and human BACE2(1-460):Fc (huBACE2:Fc) are transiently expressed in HEK293 cells. 250 μg cDNA of each construct are mixed with Fugene 6 and added to 1 liter HEK293 cells. Four days after the transfection, conditioned media are harvested for purification. huBACE1:Fc and huBACE2:Fc are purified by Protein A chromatography as described below. The enzymes are stored at −80° C. in small aliquots. (See Yang, et. al., J. Neurochemistry, 91(6) 1249-59 (2004).

### Purification of huBACE1:Fc and huBACE2:Fc

Conditioned media of HEK293 cell transiently transfected with huBACE1:Fc or huBACE2:Fc cDNA are collected. Cell debris is removed by filtering the conditioned media through 0.22 μm sterile filter. 5 ml Protein A-agarose (bed volume) is added to 4 liter conditioned media. This mixture is gently stirred overnight at 4° C. The Protein A-agarose resin is collected and packed into a low-pressure chromatography column. The column is washed with 20× bed volumes of PBS at a flow rate 20 ml per hour. Bound huBACE1:Fc or huBACE2:Fc protein is eluted with 50 mM acetic acid, pH 3.6, at flow rate 20 ml per hour. 1 ml fractions of eluent are neutralized immediately with 0.5 ml 200 mM ammonium acetate, pH 6.5. The purity of final product is assessed by electrophoresis in 4-20% Tris-Glycine SDS-PAGE. The enzyme is stored at −80° C. in small aliquots.

### BACE1 FRET Assay

Serial dilutions of the test compound are prepared as described above. The compound is further diluted 20× in KH₂PO₄ buffer. Ten μL of each dilution is added to each well on row A to H of a corresponding low protein binding black plate containing the reaction mixture (25 μL of 50 mM KH₂PO₄, pH 4.6, 1 mM TRITON® X-100, 1 mg/mL BSA, and 15 μM of FRET substrate based upon the sequence of APP) (See Yang, et. al., J. Neurochemistry, 91(6) 1249-59 (2004)). The content is mixed well on a plate shaker for 10 minutes. Fifteen μL of two hundred pM human BACE1(1-460):Fc (See Vasser, et al., Science, 286, 735-741 (1999)) in the KH₂PO₄ buffer is added to the plate containing substrate and the test compound to initiate the reaction. The RFU of the mixture at time 0 is recorded at excitation wavelength 355 nm and emission wavelength 460 nm, after brief mixing on a plate shaker. The reaction plate is covered with aluminum foil and kept in a dark humidified oven at room temperature for 16 to 24 hours. The RFU at the end of incubation is recorded with the same excitation and emission settings used at time 0. The difference of the RFU at time 0 and the end of incubation is representative of the activity of BACE1 under the compound treatment. RFU differences are plotted versus inhibitor concentration and a curve is fitted with a four-parameter logistic equation to obtain the IC₅₀ value. (May, et al., Journal of Neuroscience, 31, 16507-16516 (2011)).

The compound of Example 1 herein is tested essentially as described above and exhibits an IC₅₀ for BACE1 of 1.19 nM±0.48, n=4 (Mean±SEM; SEM=standard error of the mean). This data demonstrates that the compound of Example 1 inhibits purified recombinant BACE1 enzyme activity in vitro.

### BACE2 TMEM27 FRET Assay

Transmembrane protein 27 (TMEM27) (Accession Number NM_020665), also known as Collectrin) is a recently described substrate for BACE2, but not BACE1 (Esterhazy, et al, Cell Metabolism, 14, 365-377 (2011)). To evaluate the test compound for inhibition of BACE2 enzymatic activity, a FRET peptide (dabcyl-QTLEFLKIPS-LucY) based upon the amino acid sequence of human TMEM27 is used as a substrate (Esterhazy, et al, Cell Metabolism, 14, 365-377 (2011)). Serial dilutions of the test compound are prepared as described above. The compound is further diluted 20× in KH₂PO₄ buffer. Ten μL of each dilution is added to each well on row A to H of a corresponding low protein binding black plate containing the reaction mixture (25 μL of 50 mM KH₂PO₄, pH 4.6, 1 mM TRITON® X-100, 1 mg/mL BSA, and 5 μM of TMEM FRET substrate). Fifteen μL of twenty μM human BACE2 (1-460):Fc (See Vasser, et al., Science, 286, 735-741 (1999)) in KH₂PO₄ buffer is then added to the plate containing substrate and the test compound to initiate the reaction. The content is mixed well on a plate shaker for 10 minutes. The RFU of the mixture at time 0 is recorded at excitation wavelength 430 nm and emission wavelength 535 nm. The reaction plate is covered with aluminum foil and kept in a dark humidified oven at room temperature for 16 to 24 hours. The RFU at the end of incubation is recorded with the same excitation and emission settings used at time 0. The difference of the RFU at time 0 and the end of incubation is representative of the activity of BACE2 under the compound treatment. RFU differences are plotted versus inhibitor concentration and a curve is fitted with a four-parameter logistic equation to obtain the IC₅₀ value. (May, et al., Journal of Neuroscience, 31, 16507-16516 (2011)).

The compound of Example 1 herein is tested essentially as described above and exhibits a BACE2 IC₅₀ of 479 nM±202, n=4 (Mean±SEM; SEM=standard error of the mean). The ratio of BACE1 (FRET IC₅₀ enzyme assay) to BACE2 (TMEM27 FRET IC₅₀ assay) is about 400-fold, indicating functional selectivity for inhibiting the BACE1 enzyme. The data set forth above demonstrates that the compound of Example 1 is selective for BACE1 over BACE2.

### SH-SY5YAPP695Wt Whole Cell Assay

The routine whole cell assay for the measurement of inhibition of BACE1 activity utilizes the human neuroblastoma cell line SH-SY5Y (ATCC Accession No. CRL2266) stably expressing a human APP695Wt cDNA. Cells are routinely used up to passage number 6 and then discarded.

SH-SY5YAPP695Wt cells are plated in 96 well tissue culture plates at 5.0×10⁴ cells/well in 200 μL culture media (50% MEM/EBSS and Ham's F12, 1× each sodium pyruvate, non-essential amino acids and Na bicarbonate containing 10% FBS). The following day, media is removed from the cells, fresh media added then incubated at 37° C. for 24 hours in the presence/absence of test compound at the desired concentration range.

At the end of the incubation, conditioned media are analyzed for evidence of beta-secretase activity by analysis of Abeta peptides 1-40 and 1-42 by specific sandwich ELISAs. To measure these specific isoforms of Abeta, monoclonal 2G3 is used as a capture antibody for Abeta 1-40 and monoclonal 21F12 as a capture antibody for Abeta 1-42. Both Abeta 1-40 and Abeta 1-42 ELISAs use biotinylated 3D6 as the reporting antibody (for description of antibodies, see Johnson-Wood, et al., Proc. Natl. Acad. Sci. USA 94, 1550-1555 (1997)). The concentration of Abeta released in the conditioned media following the compound treatment corresponds to the activity of BACE1 under such conditions. The 10-point inhibition curve is plotted and fitted with the four-parameter logistic equation to obtain the IC₅₀ values for the Abeta-lowering effect. The compound of Example 1 is tested essentially as described above and exhibits the following activity for Abeta-lowering as shown in table 4.

TABLE 4

| | SH-SY5YAPP695Wt | SH-SY5YAPP695Wt |
|---|---|---|
| | A-beta (1-40) ELISA | A-beta (1-42) ELISA |
| Example # | IC₅₀ (nM) | IC₅₀ (nM) |
| 1 | 0.385 ± 0.163, n = 4 | 0.381 ± 0.266, n = 4 |

(Mean ± SEM; SEM = standard error of the mean)

### In Vivo Inhibition of Beta-Secretase

Several animal models, including mouse, guinea pig, dog, and monkey, may be used to screen for inhibition of beta-secretase activity in vivo following compound treatment. Animals used in this invention can be wild type, transgenic, or gene knockout animals. For example, the PDAPP mouse model, prepared as described in Games et al., Nature 373, 523-527 (1995), and other non-transgenic or gene knockout animals are useful to analyze in vivo inhibition of Abeta and sAPPbeta production in the presence of inhibitory compounds. Generally, 2 month old PDAPP mice, gene knockout mice or non-transgenic animals are administered compound formulated in vehicles, such as corn oil, beta-cyclodextran, phosphate buffers, PHARMASOLVE®, or other suitable vehicles via oral, subcutaneous, intra-venous, feeding, or other route of administration. One to twenty-four hours following the administration of compound, animals are sacrificed, and brains are removed for analysis of Abeta 1-x. “Abeta 1-x” as used herein refers to the sum of Abeta species that begin with residue 1 and end with a C-terminus greater than residue 28. This detects the majority of Abeta species and is often called “total Abeta”. Total Abeta peptides (Abeta 1-x) levels are measured by a sandwich ELISA, using monoclonal 266 as a capture antibody and biotinylated 3D6 as reporting antibody. (See May, et al., Journal of Neuroscience, 31, 16507-16516 (2011)).

For acute studies, compound or appropriate vehicle is administered and animals are sacrificed at about 3 hours after dosing. Brain tissue, is obtained from selected animals and analyzed for the presence of Abeta 1-x. After chronic dosing brain tissues of older APP transgenic animals may also be analyzed for the amount of beta-amyloid plaques following compound treatment.

Animals (PDAPP or other APP transgenic or non-transgenic mice) administered an inhibitory compound may demonstrate the reduction of Abeta in brain tissues, as compared with vehicle-treated controls or time zero controls. For example, a 3, 10, and 30 mg/kg oral dose of Example 1, to young female PDAPP mice reduced Abeta 1-x peptide levels in brain hippocampus by 23% (non-significant), 43% (p<0.05), and 58% (p<0.01), respectively. In brain cortical tissue, doses of 3, 10, and 30 mg/kg of Example 1 reduced Abeta 1-x levels by 43%, 59%, and 73% (all values p<0.01) compared to vehicle-treated mice three hours after dosing.

Given the activity of the Example 1, against the BACE1 enzyme in vitro, these Abeta-lowering effects are consistent with BACE inhibition in vivo, and further demonstrate CNS penetration of Example 1.

### Example 2

### Expression and Purification of Engineered N3pGlu Aβ Antibodies

Anti-N3pGlu Aβ antibodies (Antibody I or II) of the present invention can be expressed and purified essentially as follows. A glutamine synthetase (GS) expression vector containing the DNA sequence encoding the LC amino acid sequence of SEQ ID NO: 12 or 13 and the DNA sequence encoding the HC amino acid sequence of SEQ ID NO: 11 is used to transfect a Chinese hamster ovary cell line (CHO) by electroporation. The expression vector encodes an SV Early (Simian Virus 40E) promoter and the gene for GS. Post-transfection, cells undergo bulk selection with 0-50 μM L-methionine sulfoximine (MSX). Selected bulk cells or master wells are then scaled up in serum-free, suspension cultures to be used for production.

Clarified medium, into which the antibody has been secreted, is applied to a Protein A affinity column that has been equilibrated with a compatible buffer, such as phosphate buffered saline (pH 7.4). The column is washed with 1M NaCl to remove nonspecific binding components. The bound anti-N3pGlu Aβ antibody is eluted, for example, with sodium citrate at pH (approx.) 3.5 and fractions are neutralized with 1M Tris buffer. Anti-N3pGlu Aβ antibody fractions are detected, such as by SDS-PAGE or analytical size-exclusion, and then are pooled. Anti-N3pGlu Aβ antibody (Antibody I or Antibody II) of the present invention is concentrated in either PBS buffer at pH 7.4 or 10 mM NaCitrate buffer, 150 mM NaCl at pH around 6. The final material can be sterile filtered using common techniques. The purity of the anti-N3pGlu Aβ antibody is greater than 95%. The anti-N3pGlu Aβ antibody (Antibody I or Antibody II) of the present invention may be immediately frozen at −70° C. or stored at 4° C. for several months.

### Binding Affinity and Kinetics

The binding affinity and kinetics of an anti-N3pGlu Aβ antibody (Antibody I or Antibody II) to pE3-42 Aβ peptide or to Aβ 1-40 peptide is measured by surface plasmon resonance using BIACORE® 3000 (GE Healthcare). The binding affinity is measured by capturing the anti-N3pGlu Aβ antibody via immobilized protein A on a BIACORE® CMS chip, and flowing pE3-42 Aβ peptide or Aβ 1-40 peptide, starting from 100 nM in 2-fold serial dilution down to 3.125 nM. The experiments are carried out at 25° C. in HBS-EP buffer (GE Healthcare BR100669; 10 mM HEPES, 150 mM NaCl, 3 mM EDTA, 0.05% surfactant P20, pH 7.4).

For each cycle, the antibody is captured with 5 μL injection of antibody solution at a 10 μg/mL concentration with 10 μL/min. flow rate. The peptide is bound with 250 μL injection at 50 μL/min, and then dissociated for 10 minutes. The chip surface is regenerated with 5 μL injection of glycine buffer at pH 1.5 at 10 μL/mL flow rate. The data is fit to a 1:1 Langmiur binding model to derive kₒₙ, k_{off}, and to calculate K_{D}. Following procedures essentially as described above, the following parameters (shown in Table 2) were observed.

TABLE 2
Binding affinity and kinetics.

| Antibody | kₒₙ (×10⁵ 1/MS) | k_{off} (×10⁻⁴ 1/S) | K_{D} (nM) |
|---|---|---|---|
| I | 1.39 | 1.31 | 0.71 |
| II | 3.63 | 1.28 | 0.35 |

No appreciable binding to Aβ 1-40 was detected, indicating that Antibody I and Antibody II bound specifically to pE3-42 Aβ peptide as compared to Aβ 1-40.

### Ex Vivo Target Engagement

To determine ex vivo target engagement on brain sections from a fixed PDAPP brain, immunohistochemical analysis is performed with an exogenously added anti-N3pGlu Aβ antibody (Antibody I or Antibody II). Cryostat serial coronal sections from aged PDAPP mice (25-month old) are incubated with 20 μg/mL of an exemplified N3pGlu Aβ antibody of the present invention (Antibody I or Antibody II). Secondary HRP reagents specific for human IgG are employed and the deposited plaques are visualized with DAB-Plus (DAKO). Biotinylated murine 3D6 antibody followed by Step-HRP secondary is used as a positive control. The positive control antibody (biotinylated 3D6) labeled significant quantities of deposited Aβ in the PDAPP hippocampus, and the anti-N3pGlu Aβ antibodies (Antibody I or Antibody II) labeled a subset of deposits. These histological studies demonstrated that the anti-N3pGlu Aβ antibodies (Antibody I and Antibody II) engaged deposited Aβ target ex vivo.

The following Examples and assays demonstrate how a study could be designed to verify (in animal models) that the combination of antibodies of the present invention, in combination with the compound outlined herein, may be useful for treating a disease characterized by deposition of Aβ, such as of Alzheimer's disease, Down's syndrome, and CAA. It should be understood however, that the following descriptions are set forth by way of illustration and not limitation, and that various modifications may be made by one of ordinary skill in the art.

### Combination Study

### BACE Inhibitor Feeding Pilot Study

A pilot pharmacokinetic and pharmacodynamic study is performed in PDAPP mice fed a chow diet containing a BACE inhibitor, such as N-[3-[(4aS,5S,7aS)-2-amino-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide, or pharmaceutically acceptable salt thereof, in order to define doses that provide minimal to marked plasma and brain Abeta reduction by BACE inhibition alone. Young PDAPP mice are fed for 14 days a diet containing a chow diet containing the BACE inhibitor at “quasi-bid” equivalent doses of 3 mg/kg, 10 mg/kg, 30 mg/kg, or 100 mg/kg. The BACE inhibitor at ˜0.05, 0.15, 0.5, or 1.5 mg per gram of certified rodent diet #8728CM (Harlan labs) is mixed in a Sorvall mixer for 10 minutes and then mixed with Hobart mixer for 15 minutes prior to pelleting. Thirty-two young female PDAPP mice are randomized by parental line into 4 groups of 8 consisting of a vehicle-treatment group and the three doses of BACE inhibitor. Mice are allowed ad libitum access to food for 14 days and subsequently sacrificed. Mice are anesthetized with CO₂ and blood collected by cardiac puncture into EDTA-coated microcentrifuge tubes and stored on ice. Subsequently, plasma is collected by centrifugation of blood samples for 4 minutes at 14,000 rpm at room temperature, transferred to untreated microcentrifuge tubes, then frozen on dry ice and stored at −80° C. until analysis. Mice are sacrificed by decapitation, brains are rapidly micro-dissected into halves, flash frozen on dry ice and stored at −80° C. until analysis (one half for Abeta analysis and the other half for compound exposures measurement). For analysis of parenchymal Abeta, brain samples are homogenized in 5.5 M guanidine-HCl buffer (0.5 mL per half brain) with tissue tearer (model 985-370) at speed 5 for about 1 minute. Homogenized brain samples are nutated overnight at room temperature.

For Abeta ELISA analysis, extracts are collected and diluted at least 1:10 in casein buffer (1×PBS with 0.25% casein, 0.05% Tween 20, 0.1% thimerosal, pH 7.4 with protease inhibitor cocktail (Sigma P9340 at 0.01 mg/mL)) and centrifuged at 14000 rpm for 10 minutes. For analysis of plasma Abeta, samples are diluted 1:2 in specimen buffer (PBS; 0.05% Triton X-405; 0.04% thimerasol, 0.6% BSA), prior to analysis by ELISA. Plasma human Abeta₁₋ₓ is determined by sandwich ELISA using m266.2 (anti-Abeta₁₃₋₂₈) and biotinylated 3D6 (anti-Abeta1-5) as the capture and reporter antibodies, respectively. Unknowns are assayed in duplicate and pg/mL determined by interpolating (Soft Max Pro v. 5.0.1, Molecular Dynamics, using 4-parameter fit of the reference curve) from 8 point standard curves and then adjusting for dilution. Parenchymal Abeta is determined by sandwich ELISAs as described above and the values are normalized to protein levels (determined in duplicate by the Bradford Coomassie Plus Protein method) and expressed as pg/mg protein.

To determine the tissue and plasma levels of the BACE inhibitor, the following method is employed: A 0.1 mg/mL stock solution of BACE inhibitor is serially diluted with methanol/water (1:1, v/v), to prepare working solutions, which are then used to fortify control plasma and brain homogenates to yield analyte concentrations of 1, 5, 10, 20, 50, 100, 500, 1000, 2000, 4000, and 5000 ng/mL. Prior to analysis, brain samples are homogenized in 3-volumes of methanol/water (1:4, v/v) with an ultrasonic disrupter. An aliquot of each study sample, appropriate calibration standard and control matrix samples are transferred to a 96-well plate and then mixed with acetonitrile containing internal standard. After mixing, the samples are centrifuged to pellet the precipitated proteins. Aliquots of the resulting supernatants are then transferred to a clean 96-well plate and diluted with methanol/water (1:1, v/v), and 10 microliter aliquots are analyzed by LC-MS/MS. Analyte concentrations are calculated using the response to concentration relationship determined by multiple regression of the calibration curve samples.

### In Vivo Combination Study

In order to evaluate combinational plaque lowering therapy of an anti-N3pGlu Abeta antibody such as hE8L, Antibody I or Antibody II and a BACE inhibitor, such as N-[3-[(4aS,5S,7aS)-2-amino-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide, or a pharmaceutically acceptable salt thereof, a large cohort of PDAPP mice are first aged to 16 to 18-months of age. The aged PDAPP mice are randomized into five treatment arms based upon gender, parental line, and age. There are 20 to 30 aged PDAPP mice per treatment arm. Group 1 is sacrificed as a time zero at study initiation in order to determine the baseline level of pathology prior to therapeutic treatment (necropsy described below). The four remaining groups are then treated as follows: Group-2, control animals receiving placebo chow diet and weekly injections of 12.5 mg/kg of control isotype IgG2a antibody; Group-3, animals receiving weekly injections of 12.5 mg/kg anti-N3pGlu-Abeta antibody; Group-4, animals receiving BACE inhibitor chow diet at doses previously defined in the pilot feeding study, but typically ˜3 to 30 mg/kg/day; Group-5, animals receiving BACE inhibitor chow diet (˜3 to 30 mg/kg/day) and weekly injections of 12.5 mg/kg of anti-N3pGlu-Abeta antibody. The anti-N3pGlu-Abeta antibody is diluted from sterile stock solutions consisting of the antibody in PBS buffer and is administered to the animals by intraperitoneal injections. The BACE inhibitor is mixed with loose chow diet (˜0.15 to 1.5 mg compound per gram of feed depending upon desired dose) and compressed into feed pellets. Animal weight is recorded at study initiation and subsequently weekly for the first month of treatment, and then monthly for the study duration. The food intake is also monitored over the course of the study at regular intervals. The animals receive the study treatments for a total of 4-months. The animals stay on their respective diets until necropsy, which occurs one week after the final antibody injections. At time of necropsy, the animals are anesthetized and blood obtained by cardiac puncture using EDTA pre-rinsed 1 ml syringes. The blood samples are collected on ice and the plasma isolated by standard centrifugation. Subsequently, the animals are perfused with cold heparinized saline and the brain removed and dissected into the left and right hemi-spheres. One brain hemi-sphere is flash frozen and saved for histological analyses. The remaining brain hemi-sphere is dissected into tissue segments consisting of hippocampus, cortex, cerebellum, and mid-brain and subsequently frozen on dry ice. The plasma and tissue samples are stored at −80° C. until time of analysis.

### Pharmacokinetic Evaluation

Plasma pharmacokinetics is determined on the plasma samples obtained at time of necropsy. Plasma antibody levels are determined in an antigen binding ELISA assay wherein plates are coated with antigen (Abeta_{p3→42}) and subsequently incubated with diluted plasma samples or a reference standard consisting of a serial dilution of the anti-N3pGlu antibody in assay buffer (PBS+control murine plasma). After washing the plate, the bound murine antibody was detected with an anti-murine-HRP conjugated antibody followed by color development with TMB. To determine the tissue (mid-brain) and plasma levels of the BACE inhibitor, the following method is employed: A 0.1 mg/mL stock solution of BACE inhibitor is serially diluted with methanol/water (1:1, v/v), to prepare working solutions, which are then used to fortify control plasma and brain homogenates to yield analyte concentrations of 1, 5, 10, 20, 50, 100, 500, 1000, 2000, 4000, and 5000 ng/mL. Prior to analysis, brain samples are homogenized in 3-volumes of methanol/water (1:4, v/v) with an ultrasonic disrupter. An aliquot of each study sample, appropriate calibration standard and control matrix samples are transferred to a 96-well plate and then mixed with acetonitrile containing internal standard. After mixing, the samples are centrifuged to pellet the precipitated proteins. Aliquots of the resulting supernatants are then transferred to a clean 96-well plate and diluted with methanol/water (1:1, v/v), and 10 microliter aliquots are analyzed by LC-MS/MS. Analyte concentrations are calculated using the response to concentration relationship determined by multiple regression of the calibration curve samples.

### Pharmacodynamic Evaluation

The parenchymal Abeta concentrations are determined in guanidine solubilized tissue homogenates by sandwich ELISA. Tissue extraction is performed with the bead beater technology wherein frozen tissue is extracted in 1 ml of 5.5 M guanidine/50 mM Tris/0.5× protease inhibitor cocktail at pH 8.0 in 2 ml deep well dishes containing 1 ml of siliconized glass beads (sealed plates were shaken for two intervals of 3-minutes each). The resulting tissue lysates are analyzed by sandwich ELISA for Abeta₁₋₄₀ and Abeta₁₋₄₂: bead beater samples are diluted 1:10 in 2% BSA/PBS-T and filtered through sample filter plates (Millipore). Samples, blanks, standards, quality control samples, are further diluted in 0.55 M guanidine/5 mM Tris in 2% BSA/PBST prior to loading the sample plates. Reference standard are diluted in sample diluent. Plates coated with the capture antibody 21F12 (anti-Abeta₄₂) or 2G3 (anti-Abeta₄₀) at 15 μg/ml are incubated with samples and detection is accomplished with biotinylated 3D6 (anti-Abeta₁₋ₓ) diluted in 2% BSA/PBS-T, followed by 1:20 K dilution NeutrAvidin-HRP (Pierce) in 2% BSA/PBS-T and color development with TMB (Pierce). The Abeta levels are interpolated from standard curves and the final tissue concentration is calculated as nanograms of Abeta per milligram of tissue wet weight. The percent area of the hippocampus and cortex occupied by deposited Abeta is determined histologically. Cryostat serial coronal sections (7 to 10 μm thick) are incubated with 10 μg/ml of biotinylated 3D6 (anti-Abeta₁₋ₓ) or negative control murine IgG (biotinylated). Secondary HRP reagents specific for biotin are employed and the deposited Abeta visualized with DAB-Plus (DAKO). Immunoreactive Abeta deposits are quantified in defined areas of interest within the hippocampus or cortex by analyzing captured images with Image Pro plus software (Media Cybernetics).

These studies may show that the combination therapy of an anti-N3pGlu-Abeta antibody, such as hE8L, B12L, R17L, Antibody I, or Antibody II, with a BACE inhibitor, such as N-[3-[(4aS,5S,7aS)-2-amino-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide, or a pharmaceutically acceptable salt thereof, may result in enhanced Abeta reductions relative to the individual mono-therapies.

Sequences
<SEQ ID NO: 1; PRT1; Artificial> HCDR1-Antibody
I and Antibody II
KASGYTFTDYYIN
<SEQ ID NO: 2; PRT1; Artificial> HCDR2-Antibody
I and Antibody II Antibody I and Antibody II
HCDR2 (SEQ ID NO: 2)
WINPGSGNTKYNEKFKG
<SEQ ID NO: 3; PRT1; Artificial> HCDR3-Antibody
I and Antibody II
TREGETVY
<SEQ ID NO: 4; PRT1; Artificial> LCDR1-Antibody
I and Antibody II
KSSQSLLYSRGKTYLN
<SEQ ID NO: 5; PRT1; Artificial> LCDR2-Antibody II
YAVSKLDS
<SEQ ID NO: 6; PRT1; Artificial> LCDR2-Antibody I
YDVSKLDS
<SEQ ID NO: 7; PRT1; Artificial> LCDR3-Antibody I
and Antibody II
VQGTHYPFT
<SEQ ID NO: 8; PRT1; Artificial> HCVR-Antibody I
and Antibody II
QVQLVQSGAEVKKPGSSVKVSCKASGYTFTDYYINWVRQAPGQGLEWMGW
INPGSGNTKYNEKFKGRVTITADESTSTAYMELSSLRSEDTAVYYCTREG
ETVYWGQGTLVTVSS
<SEQ ID NO: 9; PRT1; Artificial> LCVR-Antibody I
DVVMTQSPLSLPVTLGQPASISCKSSQSLLYSRGKTYLNWFQQRPGQSPR
RLIYDVSKLDSGVPDRFSGSGSGTDFTLKISRVEAEDVGVYYCVQGTHYP
FTFGQGTKLEIK
<SEQ ID NO: 10; PRT1; Artificial> LCVR-Antibody II
DIQMTQSPSTLSASVGDRVTITCKSSQSLLYSRGKTYLNWLQQKPGKAPK
LLIYAVSKLDSGVPSRFSGSGSGTEFTLTISSLQPDDFATYYCVQGTHYP
FTFGQGTKLEIK
<SEQ ID NO: 11; PRT1; Artificial> Heavy Chain-
Antibody I and Antibody II
QVQLVQSGAEVKKPGSSVKVSCKASGYTFTDYYINWVRQAPGQGLEWMGW
INPGSGNTKYNEKFKGRVTITADESTSTAYMELSSLRSEDTAVYYCTREG
ETVYWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDG
SFFLYSKLTVDKSRWQQGNVFSCSVMHEALHNHYTQKSLSLSPG
<SEQ ID NO: 12; PRT1; Artificial> Light Chain-
Antibody I
DVVMTQSPLSLPVTLGQPASISCKSSQSLLYSRGKTYLNWFQQRPGQSPR
RLIYDVSKLDSGVPDRFSGSGSGTDFTLKISRVEAEDVGVYYCVQGTHYP
FTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
<SEQ ID NO: 13; PRT1; Artificial> Light Chain-
Antibody II
DIQMTQSPSTLSASVGDRVTITCKSSQSLLYSRGKTYLNWLQQKPGKAPK
LLIYAVSKLDSGVPSRFSGSGSGTEFTLTISSLQPDDFATYYCVQGTHYP
FTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
<SEQ ID NO: 14; DNA; Artificial> Exemplified DNA
for Expressing Antibody Heavy
Chain of SEQ ID NO: 11
CAGGTGCAGCTGGTGCAGTCTGGGGCTGAGGTGAAGAAGCCTGGGTCCTC
GGTGAAGGTCTCCTGCAAGGCTTCTGGATACACCTTCACCGACTATTATA
TCAACTGGGTGCGACAGGCCCCTGGACAAGGGCTTGAGTGGATGGGATGG
ATCAACCCTGGCAGTGGTAATACAAAGTACAATGAGAAGTTCAAGGGCAG
AGTCACGATTACCGCGGACGAATCCACGAGCACAGCCTACATGGAGCTGA
GCAGCCTGAGATCTGAGGACACGGCCGTGTATTACTGTACAAGAGAAGGC
GAGACGGTCTACTGGGGCCAGGGAACCCTGGTCACCGTCTCCTCAGCCTC
CACCAAGGGCCCATCGGTCTTCCCGCTAGCACCCTCCTCCAAGAGCACCT
CTGGGGGCACAGCGGCCCTGGGCTGCCTGGTCAAGGACTACTTCCCCGAA
CCGGTGACGGTGTCGTGGAACTCAGGCGCCCTGACCAGCGGCGTGCACAC
CTTCCCGGCTGTCCTACAGTCCTCAGGACTCTACTCCCTCAGCAGCGTGG
TGACCGTGCCCTCCAGCAGCTTGGGCACCCAGACCTACATCTGCAACGTG
AATCACAAGCCCAGCAACACCAAGGTGGACAAGAAAGTTGAGCCCAAATC
TTGTGACAAAACTCACACATGCCCACCGTGCCCAGCACCTGAACTCCTGG
GGGGACCGTCAGTCTTCCTCTTCCCCCCAAAACCCAAGGACACCCTCATG
ATCTCCCGGACCCCTGAGGTCACATGCGTGGTGGTGGACGTGAGCCACGA
AGACCCTGAGGTCAAGTTCAACTGGTACGTGGACGGCGTGGAGGTGCATA
ATGCCAAGACAAAGCCGCGGGAGGAGCAGTACAACAGCACGTACCGTGTG
GTCAGCGTCCTCACCGTCCTGCACCAGGACTGGCTGAATGGCAAGGAGTA
CAAGTGCAAGGTCTCCAACAAAGCCCTCCCAGCCCCCATCGAGAAAACCA
TCTCCAAAGCCAAAGGGCAGCCCCGAGAACCACAGGTGTACACCCTGCCC
CCATCCCGGGACGAGCTGACCAAGAACCAGGTCAGCCTGACCTGCCTGGT
CAAAGGCTTCTATCCCAGCGACATCGCCGTGGAGTGGGAGAGCAATGGGC
AGCCGGAGAACAACTACAAGACCACGCCCCCCGTGCTGGACTCCGACGGC
TCCTTCTTCCTCTATAGCAAGCTCACCGTGGACAAGAGCAGGTGGCAGCA
GGGGAACGTCTTCTCATGCTCCGTGATGCATGAGGCTCTGCACAACCACT
ACACGCAGAAGAGCCTCTCCCTGTCTCCGGGT
<SEQ ID NO: 15; DNA; Artificial> Exemplified DNA
for Expressing Antibody Light Chain of SEQ ID
NO: 12
GATGTTGTGATGACTCAGTCTCCACTCTCCCTGCCCGTCACCCTTGGACA
GCCGGCCTCCATCTCCTGCAAGTCTAGTCAAAGCCTCCTGTACAGTCGCG
GAAAAACCTACTTGAATTGGTTTCAGCAGAGGCCAGGCCAATCTCCAAGG
CGCCTAATTTATGATGTTTCTAAACTGGACTCTGGGGTCCCAGACAGATT
CAGCGGCAGTGGGTCAGGCACTGATTTCACACTGAAAATCAGCAGGGTGG
AGGCTGAGGATGTTGGGGTTTATTACTGCGTGCAAGGTACACACTACCCT
TTCACTTTTGGCCAAGGGACCAAGCTGGAGATCAAACGGACCGTGGCTGC
ACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAA
GTACAGTGGAAGGTGGATAACGCCCTCCAATCGGGTAACTCCCAGGAGAG
TGTCACAGAGCAGGACAGCAAGGACAGCACCTACAGCCTCAGCAGCACCC
TGACGCTGAGCAAAGCAGACTACGAGAAACACAAAGTCTACGCCTGCGAA
GTCACCCATCAGGGCCTGAGCTCGCCCGTCACAAAGAGCTTCAACAGGGG
AGAGTGC
<SEQ ID NO: 16; DNA; Artificial> Exemplified DNA
for Expressing Antibody Light Chain of SEQ ID
NO: 13
GACATCCAGATGACCCAGTCTCCTTCCACCCTGTCTGCATCTGTAGGAGA
CAGAGTCACCATCACTTGCAAGTCCAGTCAGAGTCTCCTGTACAGTCGCG
GAAAAACCTATTTGAACTGGCTCCAGCAGAAACCAGGGAAAGCCCCTAAG
CTCCTGATCTATGCTGTCTCCAAACTGGACAGTGGGGTCCCATCAAGGTT
CAGCGGCAGTGGATCTGGGACAGAATTCACTCTCACCATCAGCAGCCTGC
AGCCTGATGATTTTGCAACTTATTACTGCGTGCAGGGTACACATTATCCT
TTCACTTTTGGCCAGGGGACCAAGCTGGAGATCAAACGGACCGTGGCTGC
ACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAA
GTACAGTGGAAGGTGGATAACGCCCTCCAATCGGGTAACTCCCAGGAGAG
TGTCACAGAGCAGGACAGCAAGGACAGCACCTACAGCCTCAGCAGCACCC
TGACGCTGAGCAAAGCAGACTACGAGAAACACAAAGTCTACGCCTGCGAA
GTCACCCATCAGGGCCTGAGCTCGCCCGTCACAAAGAGCTTCAACAGGGG
AGAGTGC
<SEQ ID NO: 17; PRT1; Artificial> (LCDR1-B12L/
R17L/hE8L)
KSSQSLLYSRGKTYLN
<SEQ ID NO: 18; PRT1; Artificial> (LCDR2-B12L/
R17L/hE8L)
AVSKLDS
<SEQ ID NO: 19; PRT1; Artificial> (LCDR3-B12L/
R17L/hE8L)
VQGTHYPFT
<SEQ ID NO: 20; PRT1; Artificial> (HCDR1-B12L)
GYDFTRYYIN
<SEQ ID NO: 21; PRT1; Artificial> (HCDR1-R17L)
GYTFTRYYIN
<SEQ ID NO: 22; PRT1; Artificial> (HCDR2-B12L/
R17L/hE8L)
WINPGSGNTKYNEKFKG
<SEQ ID NO: 23; PRT1; Artificial> (HCDR3-B12L)
EGITVY
<SEQ ID NO: 24; PRT1; Artificial> (HCDR3-R17L)
EGTTVY
<SEQ ID NO: 25; PRT1; Artificial> (LCVR-B12L/R17L)
DIVMTQTPLSLSVTPGQPASISCKSSQSLLYSRGKTYLNWLLQKPGQSPQ
LLIYAVSKLDSGVPDRFSGSGSGTDFTLKISRVEAEDVGVYYCVQGTHYP
FTFGQGTKLEIK
<SEQ ID NO: 26; PRT1; Artificial> (HCVR-B12L)
QVQLVQSGAEVKKPGSSVKVSCKASGYDFTRYYINWVRQAPGQGLEWMGW
INPGSGNTKYNEKFKGRVTITADESTSTAYMELSSLRSEDTAVYYCAREG
ITVYWGQGTTVTVSS
<SEQ ID NO: 27; PRT1; Artificial> (HCVR-R17L)
QVQLVQSGAEVKKPGSSVKVSCKASGYTFTRYYINWVRQAPGQGLEWMGW
INPGSGNTKYNEKFKGRVTITADESTSTAYMELSSLRSEDTAVYYCAREG
TTVYWGQGTTVTVSS
<SEQ ID NO: 28; PRT1; Artificial> (LC-B12L/R17L)
DIVMTQTPLSLSVTPGQPASISCKSSQSLLYSRGKTYLNWLLQKPGQSPQ
LLIYAVSKLDSGVPDRFSGSGSGTDFTLKISRVEAEDVGVYYCVQGTHYP
FTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
<SEQ ID NO: 29; PRT1; Artificial> (HC-B12L)
QVQLVQSGAEVKKPGSSVKVSCKASGYDFTRYYINWVRQAPGQGLEWMGW
INPGSGNTKYNEKFKGRVTITADESTSTAYMELSSLRSEDTAVYYCAREG
ITVYWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDG
SFFLYSKLTVDKSRWQQGNVFSCSVMHEALHNHYTQKSLSLSPG
<SEQ ID NO: 30; PRT1; Artificial> (HC-R17L)
QVQLVQSGAEVKKPGSSVKVSCKASGYTFTRYYINWVRQAPGQGLEWMGW
INPGSGNTKYNEKFKGRVTITADESTSTAYMELSSLRSEDTAVYYCAREG
TTVYWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDG
SFFLYSKLTVDKSRWQQGNVFSCSVMHEALHNHYTQKSLSLSPG
N3pGlu Aβ
(SEQ ID NO: 31)
[pE]FRHDSGYEVHHQKLVFFAEDVGSNKGAIIGLMVGGVVIA
<SEQ ID NO, 32; PRTl; Artificial> (LCVR-hE8L)
DIVMTQTPLSLSVTPGQPASISCKSSQSLLYSRGKTYLNWLLQKPGQSPQ
LLIYAVSKLDSGVPDRFSGSGSGTDFTLKISRVEAEDVGVYYCVQGTHYP
FTFGQGTKLEIK
<SEQ ID NO, 33; PRT1; Artificial> (LC-hE8L)
DIVMTQTPLSLSVTPGQPASISCKSSQSLLYSRGKTYLNWLLQKPGQSPQ
LLIYAVSKLDSGVPDRFSGSGSGTDFTLKISRVEAEDVGVYYCVQGTHYP
FTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
<SEQ ID NO, 34; PRTl; Artificial> (HCVR-hE8L)
QVQLVQSGAEVKKPGSSVKVSCKASGYTFTDYYINWVRQAPGQGLEWMGW
INPGSGNTKYNEKFKGRVTITADESTSTAYMELSSLRSEDTAVYYCAREG
ETVYWGQGTTVTVSS
<SEQ ID NO, 35; PRTl; Artificial> (HC-hE8L)
QVQLVQSGAEVKKPGSSVKVSCKASGYTFTDYYINWVRQAPGQGLEWMGW
INPGSGNTKYNEKFKGRVTITADESTSTAYMELSSLRSEDTAVYYCAREG
ETVYWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDG
SFFLYSKLTVDKSRWQQGNVFSCSVMHEALHNHYTQKSLSLSPG
<SEQ ID NO: 36; PRT1; Artificial> (HCDR1-hE8L)
GYTFTDYYIN
<SEQ ID NO: 37; PRT1; Artificial> (HCDR3-hE8L)
EGETVY
<SEQ ID NO: 38; PRT1; Artificial> (Aβ 1-42)
DAEFRHDSGYEVHHQKLVFFAEDVGSNKGAIIGLMVGGVVIA

1. A method of treating Alzheimer's disease, comprising administering to a patient in need of such treatment an effective amount of a compound of the formula: 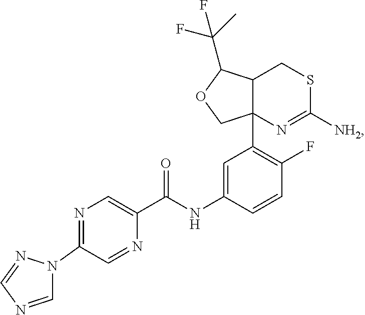 [SMILES: CC(F)(F)C1OCC2(c3cc(NC(=O)c4cnc(-n5cncn5)cn4)ccc3F)N=C(N)SCC12]
or a pharmaceutically acceptable salt thereof, in combination with an effective amount of anti-N3pGlu Abeta antibody wherein the anti-N3pGlu Abeta antibody antibody comprises a light chain variable region (LCVR) and a heavy chain variable region (HCVR), wherein said LCVR comprises LCDR1, LCDR2 and LCDR3 and HCVR comprises HCDR1, HCDR2 and HCDR3 which are selected from the group consisting of:
a) LCDR1 is SEQ ID. NO: 17, LCDR2 is SEQ ID. NO: 18, LCDR3 is SEQ ID. NO: 19, HCDR1 is SEQ ID. NO: 20, HCDR2 is SEQ ID: NO: 22, and HCDR3 is SEQ ID. NO: 23; and
b) LCDR1 is SEQ ID. NO: 17, LCDR2 is SEQ ID. NO: 18, LCDR3 is SEQ ID. NO: 19, HCDR1 is SEQ ID. NO: 21, HCDR2 is SEQ ID. NO: 22, and HCDR3 is SEQ ID. NO: 24;
c) LCDR1 is SEQ ID. NO: 17, LCDR2 is SEQ ID. NO: 18, LCDR3 is SEQ ID. NO: 19, HCDR1 is SEQ ID. NO: 36, HCDR2 is SEQ ID. NO: 22, and HCDR3 is SEQ ID. NO: 37;
d) LCDR1 is SEQ ID. NO: 4, LCDR2 is SEQ ID. NO: 6, LCDR3 is SEQ ID. NO: 7, HCDR1 is SEQ ID. NO: 1, HCDR2 is SEQ ID. NO: 2, and HCDR3 is SEQ ID. NO: 3;
e) LCDR1 is SEQ ID. NO: 4, LCDR2 is SEQ ID. NO: 5, LCDR3 is SEQ ID. NO: 7, HCDR1 is SEQ ID. NO: 1, HCDR2 is SEQ ID. NO: 2, and HCDR3 is SEQ ID. NO: 3.

2. The method according to claim 1 wherein the compound is N-[3-[(4aS,5S,7aS)-2-amino-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide, or a pharmaceutically acceptable salt thereof.

3. The method according claim 2, wherein the anti-N3pGlu Abeta antibody comprises a light chain variable region (LCVR) and a heavy chain variable region (HCVR), wherein said LCVR and HCVR are selected from the group consisting of
a) LCVR of SEQ ID NO: 25 and HCVR of SEQ ID NO: 26;
b) LCVR of SEQ ID NO: 25 and HCVR of SEQ ID NO: 27;
c) LCVR of SEQ ID NO: 32 and HCVR of SEQ ID NO: 34;
d) LCVR of SEQ ID NO: 9 and HCVR of SEQ ID NO: 8; and
e) LCVR of SEQ ID NO: 10 and HCVR of SEQ ID NO: 8.

4. The method according to claim 3, wherein the anti-N3pGlu Abeta antibody comprises a light chain (LC) and a heavy chain (HC), wherein said LC and HC are selected from the group consisting of
a) LC of SEQ ID NO: 28 and HC of SEQ ID NO: 29;
b) LC of SEQ ID NO: 28 and HC of SEQ ID NO: 30;
c) LC of SEQ ID NO: 33 and HC of SEQ ID NO: 35;
d) LC of SEQ ID NO: 12 and HC of SEQ ID NO: 11; and
e) LC of SEQ ID NO: 13 and HC of SEQ ID NO: 11.

5. The method according to claim 4, wherein the anti-N3pGlu Abeta antibody comprises two light chains (LC) and two heavy chains (HC), wherein each LC and each HC are selected from the group consisting of
a) LC of SEQ ID NO: 28 and HC of SEQ ID NO: 29;
b) LC of SEQ ID NO: 28 and HC of SEQ ID NO: 30;
c) LC of SEQ ID NO: 33 and HC of SEQ ID NO: 35;
d) LC of SEQ ID NO: 12 and HC of SEQ ID NO: 11; and
e) LC of SEQ ID NO: 13 and HC of SEQ ID NO: 11.

6. The method according to claim 5 wherein the compound and the anti-N3pGlu Abeta antibody are administered simultaneously

7. The method according to according to claim 5 wherein the compound is administered prior to the administration of the anti-N3pGlu Abeta antibody.

8. The method according to claim 5, wherein the anti-N3pGlu Abeta antibody comprises an LC of SEQ ID NO: 28 and HC of SEQ ID NO: 29.

9. The method according to claim 5, wherein the anti-N3pGlu Abeta antibody comprises an LC of SEQ ID NO: 28 and HC of SEQ ID NO: 30.

10. The method according to claim 5, wherein the anti-N3pGlu Abeta antibody comprises an LC of SEQ ID NO: 33 and HC of SEQ ID NO: 35.

11. The method according to claim 5, wherein the anti-N3pGlu Abeta antibody comprises an LC of SEQ ID NO: 12 and HC of SEQ ID NO: 11.

12. The method according to claim 5, wherein the anti-N3pGlu Abeta antibody comprises an LC of SEQ ID NO: 13 and HC of SEQ ID NO: 11.

13. (canceled)

14. (canceled)

15. (canceled)

16. (canceled)

17. (canceled)

18. (canceled)

19. (canceled)

20. (canceled)

21. (canceled)

22. (canceled)

23. A pharmaceutical composition, comprising a compound N-[3-[(4aS,5S,7aS)-2-amino-5-(1,1-difluoroethyl)-4,4a,5,7-tetrahydrofuro[3,4-d][1,3]thiazin-7a-yl]-4-fluoro-phenyl]-5-(1,2,4-triazol-1-yl)pyrazine-2-carboxamide, or a pharmaceutically acceptable salt thereof, with one or more pharmaceutically acceptable carriers, diluents, or excipients, in combination with a pharmaceutical composition of anti-N3pGlu Abeta antibody, with one or more pharmaceutically acceptable carriers, diluents, or excipients.

24. The pharmaceutical composition according to claim 23, wherein the anti-N3pGlu Abeta antibody comprises a light chain variable region (LCVR) and a heavy chain variable region (HCVR), wherein said LCVR comprises LCDR1, LCDR2 and LCDR3 and HCVR comprises HCDR1, HCDR2 and HCDR3 which are selected from the group consisting of:
a) LCDR1 is SEQ ID. NO: 17, LCDR2 is SEQ ID. NO: 18, LCDR3 is SEQ ID. NO: 19, HCDR1 is SEQ ID. NO: 20, HCDR2 is SEQ ID: NO: 22, and HCDR3 is SEQ ID. NO: 23; and
b) LCDR1 is SEQ ID. NO: 17, LCDR2 is SEQ ID. NO: 18, LCDR3 is SEQ ID. NO: 19, HCDR1 is SEQ ID. NO: 21, HCDR2 is SEQ ID. NO: 22, and HCDR3 is SEQ ID. NO: 24;
c) LCDR1 is SEQ ID. NO: 17, LCDR2 is SEQ ID. NO: 18, LCDR3 is SEQ ID. NO: 19, HCDR1 is SEQ ID. NO: 36, HCDR2 is SEQ ID. NO: 22, and HCDR3 is SEQ ID. NO: 37;
d) LCDR1 is SEQ ID. NO: 4, LCDR2 is SEQ ID. NO: 6, LCDR3 is SEQ ID. NO: 7, HCDR1 is SEQ ID. NO: 1, HCDR2 is SEQ ID. NO: 2, and HCDR3 is SEQ ID. NO: 3;
e) LCDR1 is SEQ ID. NO: 4, LCDR2 is SEQ ID. NO: 5, LCDR3 is SEQ ID. NO: 7, HCDR1 is SEQ ID. NO: 1, HCDR2 is SEQ ID. NO: 2, and HCDR3 is SEQ ID. NO: 3.

25. The pharmaceutical composition according to claim 24, wherein the anti-N3pGlu Abeta antibody comprises a light chain variable region (LCVR) and a heavy chain variable region (HCVR), wherein said LCVR and HCVR are selected from the group consisting of
a) LCVR of SEQ ID NO: 25 and HCVR of SEQ ID NO: 26;
b) LCVR of SEQ ID NO: 25 and HCVR of SEQ ID NO: 27;
c) LCVR of SEQ ID NO: 32 and HCVR of SEQ ID NO: 34;
d) LCVR of SEQ ID NO: 9 and HCVR of SEQ ID NO: 8; and
e) LCVR of SEQ ID NO: 10 and HCVR of SEQ ID NO: 8.

26. The pharmaceutical composition according to claim 25, wherein the anti-N3pGlu Abeta antibody comprises a light chain (LC) and a heavy chain (HC), wherein said LC and HC are selected from the group consisting of
a) LC of SEQ ID NO: 28 and HC of SEQ ID NO: 29;
b) LC of SEQ ID NO: 28 and HC of SEQ ID NO: 30;
c) LC of SEQ ID NO: 33 and HC of SEQ ID NO: 35;
d) LC of SEQ ID NO: 12 and HC of SEQ ID NO: 11; and
e) LC of SEQ ID NO: 13 and HC of SEQ ID NO: 11.

27. The pharmaceutical composition according to claim 26, wherein the anti-N3pGlu Abeta antibody comprises two light chains (LC) and two heavy chains (HC), wherein each LC and each HC are selected from the group consisting of
a) LC of SEQ ID NO: 28 and HC of SEQ ID NO: 29;
b) LC of SEQ ID NO: 28 and HC of SEQ ID NO: 30;
c) LC of SEQ ID NO: 33 and HC of SEQ ID NO: 35;
d) LC of SEQ ID NO: 12 and HC of SEQ ID NO: 11; and
e) LC of SEQ ID NO: 13 and HC of SEQ ID NO: 11.

28. The pharmaceutical composition according to claim 27 wherein the anti-N3pGlu Abeta antibody comprises an LC of SEQ ID NO: 28 and HC of SEQ ID NO: 29.

29. The pharmaceutical composition according to claim 27 wherein the anti-N3pGlu Abeta antibody comprises an LC of SEQ ID NO: 28 and HC of SEQ ID NO: 30.

30. The pharmaceutical composition according to claim 27 wherein the anti-N3pGlu Abeta antibody comprises an LC of SEQ ID NO: 33 and HC of SEQ ID NO: 35.

31. The pharmaceutical composition according to claim 27 wherein the anti-N3pGlu Abeta antibody comprises an LC of SEQ ID NO: 12 and HC of SEQ ID NO: 11.

32. The pharmaceutical composition according to claim 27 wherein the anti-N3pGlu Abeta antibody comprises an LC of SEQ ID NO: 13 and HC of SEQ ID NO: 11.
